SEQUENCE LISTING (28 sequences)

SEQ ID NO: 1 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 2 - protein, 5 aa, Homo sapiens
DYNMH

SEQ ID NO: 3 - protein, 17 aa, Homo sapiens
YIVPLNGGSTFNQKFKG

SEQ ID NO: 4 - protein, 7 aa, Homo sapiens
GGTRFAY

SEQ ID NO: 5 - protein, 15 aa, Homo sapiens
RASESVDNFGVSFMY

SEQ ID NO: 6 - protein, 7 aa, Homo sapiens
GASNQGS

SEQ ID NO: 7 - protein, 9 aa, Homo sapiens
QQTKEVPYT

SEQ ID NO: 8 - protein, 116 aa, synthetic construct
QVQLVQSGAEVKKPGASVKVSCKASGYTFTDYNMHWVRQAPGQRLEWIGYIVPLNGGSTFNQKFKGRATITVDTSARTAYMELSSLRSEDTAVYYCARGGTRFAYWGQGTLVTVSS

SEQ ID NO: 9 - protein, 111 aa, synthetic construct
DIVMTQSPDSLAVSLGERATINCRASESVDNFGVSFMYWFQQKPGQPPKLLIYGASNQGSGVPDRFSGSGSGTDFTLTISSLQAEDVAVYYCQQTKEVPYTFGGGTKVEIK

SEQ ID NO: 10 - protein, 446 aa, synthetic construct
QVQLVQSGAEVKKPGASVKVSCKASGYTFTDYNMHWVRQAPGQRLEWIGYIVPLNGGSTFNQKFKGRATITVDTSARTAYMELSSLRSEDTAVYYCARGGTRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 218 aa, synthetic construct
DIVMTQSPDSLAVSLGERATINCRASESVDNFGVSFMYWFQQKPGQPPKLLIYGASNQGSGVPDRFSGSGSGTDFTLTISSLQAEDVAVYYCQQTKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 13 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 14 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 15 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 16 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 10 aa, synthetic construct
GFTFSRYWMS

SEQ ID NO: 19 - protein, 17 aa, synthetic construct
NIKQDGSEKYYVDSVKG

SEQ ID NO: 20 - protein, 12 aa, synthetic construct
EGGWFGELAFDY

SEQ ID NO: 21 - protein, 12 aa, synthetic construct
RASQRVSSSYLA

SEQ ID NO: 22 - protein, 7 aa, synthetic construct
DASSRAT

SEQ ID NO: 23 - protein, 9 aa, synthetic construct
QQYGSLPWT

SEQ ID NO: 24 - protein, 118 aa, synthetic construct [sequence omitted]

SEQ ID NO: 25 - protein, 122 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 26 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 27 - protein, 445 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKQVSLTCLVKGFYPSDIAVEWESNGQPENYKTTPPVLDSDGSFFLYSKLTVDKSRWQQGNVFSCSVMHEALHNHYTQKSLSLSPG

SEQ ID NO: 28 - protein, 213 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0191954 A1
(43) Pub. Date: Jul. 9, 2026

(54) COMPOSITIONS AND METHODS FOR NEOADJUVANT TREATMENT IN CANCER

(71) Applicants: INNATE PHARMA, MARSEILLE (FR); MEDIMMUNE LIMITED, CAMBRIDGE (GB)

(72) Inventors: NADIA ANCERIZ, FEIGÈRES (FR); CHUNLING FAN, GAITHERSBURG, MD (US); ZACHARY COOPER, GAITHERSBURG, MD (US); CAROLINE DENIS, MARSEILLE (FR); JAMES EDWARD EYLES, CAMBRIDGE (GB); PAULA FRAENKEL, WALTHAM, MA (US); CARINE PATUREL, MARCY L'ETOILE (FR); ERIC TU, GAITHERSBURG, MD (US)

(21) Appl. No.: 19/133,798

(22) PCT Filed: Nov. 30, 2023
(86) PCT No.: PCT/IB2023/000728

§ 371 Date: May 29, 2025

Related U.S. Application Data
(60) Provisional application No. 63/385,628, filed on Dec. 1, 2022.

(51) Int. Cl. A61K 39/395; A61K 31/282; A61K 31/337; A61K 31/519; A61K 33/243; A61P 35/00

(52) U.S. Cl. CPC A61K 39/3955; A61K 31/282; A61K 31/337; A61K 31/519; A61K 33/243; A61P 35/00

(57) ABSTRACT

The present invention relates to antibodies that inhibit the enzymatic activity of human CD39 and methods of using the compounds to treat cancer, including in combination with an anti-PD(L)1 antibody. The anti-CD39 antibody and the anti-PD(L)1 antibody can be administered as neoadjuvant therapy to treat lung cancer. The invention also relates to dosing regimens for combination treatment with anti-CD39 antibody and an anti-PD(L)1 antibody.

### CROSS-REFERENCE TO RELATED APPLICATION

This application claims the benefit of U.S. Provisional Application No. 63/385,628 filed Dec. 1, 2022; which is incorporated herein by reference in its entirety; including any drawings.

### REFERENCE TO SEQUENCE LISTING

The present application is being filed along with a Sequence Listing in electronic format. The Sequence Listing is provided as a file entitled “INN.276XPCT.xml” which was created on Nov. 13, 2023 and is 28,938 bytes. The information in the electronic format of the Sequence Listing is incorporated herein by reference in its entirety.

### FIELD OF THE INVENTION

The present invention relates to antibodies that inhibit the enzymatic activity of human CD39 and methods of using the compounds to treat cancer.

### BACKGROUND

Eight different ENTPD genes encode members of the NTPDase protein family. The individual NTPDase subtypes differ in cellular location and functional properties. Plasma membrane-bound nucleoside triphosphate diphosphohydrolases control nucleotide levels at the cell surface by hydrolyzing the c and b phosphates of nucleotides. NTPDase 1 (ectonucleoside triphosphate diphosphohydrolase1), also known as CD39/ENTPD1 or vascular CD39, functions together with another enzyme, CD73 (ecto-5′-nucleotidase), to hydrolyze extracellular adenosine triphosphate (ATP) and adenosine diphosphate (ADP) to generate adenosine, which binds to adenosine receptors and inhibits T-cell and natural killer (NK)-cell responses, thereby suppressing the immune system. The generation of adenosine via the CD73/CD39 pathway is recognized as a major mechanism of regulatory T cell (Treg) immunosuppressive function. The number of CD39⁺ Tregs is increased in some human cancers, and the importance of CD39⁺ Tregs in promoting tumor growth and metastasis has been demonstrated using several in vivo models. However, CD39 is also expressed by tumor cells and CD39⁺ tumor cells can mediate immunosuppression via the adenosine pathway. CD39 in cancer cells displays ATPase activity and, together with CD73, generates adenosine. CD73⁺CD39⁺ cancer cells inhibited the proliferation of CD4 and CD8 T cells and the generation of cytotoxic effector CD8 T cells (CTL) in a CD39- and adenosine-dependent manner.

CD39 expression has been reported to be increased in several solid tumors (colorectal cancer, head and neck cancer, pancreatic cancer) as well as in chronic lymphocytic leukemia. Antibodies that bind and inhibit the enzymatic activity of CD39 are disclosed for example in WO2018/167267, WO2019/243252, WO2019/178269, WO2019/127935, and WO2021/037037.

In many cases of cancer, disease may be determined upon diagnosis to be surgically resectable or may become surgically resectable following an initial radiotherapy or chemotherapy. Surgery often offers the best chance of a cure for many tumors.

Worldwide, roughly 1.5 million new cases of lung cancer are diagnosed annually with about 85% being non-small-cell lung cancers (NSCLCs). Surgery is thought to be the best treatment option, but only about 20-30% of patients with NSCLC present with surgically resectable disease (Molina et al., Mayo Clin. Proc. 83 (5): 584-94 (2008); Burdett S. Lancet. 2014; 383:1561-1571). Adjuvant chemotherapy following resection of NSCLC is standard practice to reduce risk of disease recurrence. In cases of limited disease (stage I, II, IIIA) patients who undergo surgical resection and the administration of chemotherapy achieve a 5-year survival of 51%, with an absolute benefit of 5.4% in 5-year survival, especially in patients with a good performance status (PS) (Provencio et al. 2011 J Thorac Dis. 3 (3): 197-204). There is evidence that identification of the minimal residual disease (MRD) status of a patient through detection of circulating tumor DNA (ctDNA) post-surgery can accurately predict disease recurrence. It has been proposed that durvalumab may be particularly beneficial in a high-risk NSCLC patient population patients in which MRD is detected via ctDNA isolation after complete resection. Further, in some cases, neoadjuvant chemotherapy is used. Burdett S. Lancet. 2014; 383:1561-1571 concluded that in stage IB-IIIA, preoperative chemotherapy significantly improves overall survival, time to distant recurrence, and recurrence-free survival in resectable NSCLC. Such preoperative neoadjuvant chemotherapy has been proposed as having the potential to reduce tumour size, increase operability, and eradicate micrometastases. Neoadjuvant chemotherapy might also be more effective when the blood supply to the tumour is still intact before surgical resection, and chemotherapy might be better tolerated if patients are not recovering from major surgery. Some clinical trials have evaluated chemotherapy administered only preoperatively and five trials evaluated chemotherapy preoperatively and then postoperatively, usually to responders (see Burnett 2014, supra), however, it is concluded that the potential benefit of neoadjuvant chemotherapy would need to be balanced against possible toxic effects.

There remains a need in the art to improve the treatment of cancer. Many patients with locally advanced disease (stage III) have disease deemed unresectable on diagnosis, and it would be desirable to render the disease more operable or resectable. For patients with stage II-IIIA and select IIIB disease, despite disease being resectable, surgery and adjuvant standard of care (SoC) chemotherapy results in 5-year disease-free survival (DFS) rates of only-40% (Wakelee et al., Lancet. Oncol. 18 (12): 1610-23 (2017).

### SUMMARY OF THE INVENTION

The disclosure provides methods of treating cancer and/or preventing the recurrence of a cancer in a patient in need thereof, for example in stage I-III NSCLC, optionally stage IB-IIIA NSCLC, optionally stage II or IIIA NSCLC. The disclosure also provides dosing regimens for anti-CD39 antibody and an anti-PD(L)1 antibody that are adapted to such treatments, e.g. in resectable tumors or cancers.

The disclosure provides a method of treating a tumor or cancer and/or preventing the recurrence of a tumor or cancer in a patient in need thereof, comprising administering an anti-CD39 antibody and an anti-PD(L)1 antibody, wherein the anti-CD39 antibody and the anti-PD(L)1 antibody are administered as neoadjuvant therapy (preoperative therapy). In one embodiment, the method comprises administering an anti-CD39 antibody, the anti-PD(L)1 antibody and a chemotherapeutic agent, wherein the anti-CD39 antibody, the anti-PD(L)1 antibody and the chemotherapeutic agent are administered as neoadjuvant therapy. The disclosure further provides a method of treating a tumor or cancer and/or preventing the recurrence of a tumor or cancer in a patient in need thereof, comprising administering an anti-CD39 antibody and an anti-PD(L)1 antibody, wherein the anti-CD39 antibody and the anti-PD(L)1 antibody are administered as a neoadjuvant therapy and further as an adjuvant therapy. The treatment regimen of the disclosure can be characterized as comprising: (a) administering to the patient, before surgical tumor resection or wherein the patient has not undergone surgical resection, an anti-CD39 antibody and an anti-PD(L)1 antibody (and optionally further an effective amount of a chemotherapy); and (b) administering to the patient, after surgical tumor resection, an anti-CD39 antibody and an anti-PD(L)1 antibody. In one embodiment, the tumor or cancer is characterized as a tumor or cancer that is deemed surgically resectable. In one embodiment, the tumor or cancer is characterized as having potential to become surgically resectable (e.g. a locally advanced and/or Stage IIIB NSCLC).

In one embodiment, the anti-CD39 antibody is an antibody comprising the amino acid sequences of SEQ ID NOS: 2-7, optionally the antibody comprises the amino acid sequences of SEQ ID NOS: 8 and 9, optionally the antibody comprises the amino acid sequences of SEQ ID NOS: 10 and 11. In one embodiment, the anti-PD(L)1 antibody is durvalumab.

The methods are particularly advantageous in the treatment of lung cancer, particularly non-small cell lung cancer (NSCLC). In any embodiment, the cancer can optionally be characterized as being a resectable NSCLC. In one embodiment, the cancer is a stage II or IIIA NSCLC, e.g. a resectable stage II or IIIA NSCLC. In one embodiment, the cancer is a stage I, Il or IIIA NSCLC. In another aspect, optionally the cancer can be characterized as being an unresectable and/or locally advanced NSCLC (e.g. a stage III or IIIB NSCLC).

In one embodiment, the treatment regimens herein provide administration of the anti-CD39 antibody at the same dose (e.g. 2250 mg or 3000 mg) both in the adjuvant setting and in the neoadjuvant setting. In one embodiment, the treatment regimens herein permit administration of the anti-CD39 antibody and the anti-PD(L)1 antibody at the same respective doses (e.g. 2250 mg or 3000 mg fixed dose for the anti-CD39 antibody and 1500 mg fixed dose for durvalumab) both in the adjuvant setting and in the neoadjuvant setting. Furthermore, the regimens permit the anti-CD39 antibody (and further the anti-PD(L)1 antibody) to be administered every three weeks as neoadjuvant and every four weeks as adjuvant (at the same dosage).

In one embodiment, provided is a method of administering an anti-CD39 antibody comprising the amino acid sequences of SEQ ID NOS: 2-7, 8-9 or 10-11, wherein the antibody is administered Q3w or Q4w at a dose of 3000 mg. In one embodiment, provided is a method for administering an anti-CD39 antibody comprising the amino acid sequences of SEQ ID NOS: 2-7, 8-9 or 10-11, wherein the antibody is administered Q3w or Q4w at a dose of 2250 mg.

In one embodiment, provided is a method for administering an anti-CD39 antibody comprising the amino acid sequences of SEQ ID NOS: 2-7, 8-9 or 10-11, wherein the antibody is administered (a) on day 1 of a 3 week cycle for one or more cycles and (b) on day 1 of a 4 week cycle for one more cycles, wherein in each case the antibody is administered at a dose of 2250 or 3000 mg. Optionally, the method is a method of treating cancer or preventing recurrence of cancer. Optionally, the method is a method of administering an anti-CD39 antibody in combination with an anti-PD(L)1 antibody.

In one embodiment, provided is a method for administering an anti-CD39 antibody and an anti-PD(L)1 antibody, wherein the anti-CD39 antibody and the anti-PD(L)1 antibody are administered (a) on day 1 of a 3 week cycle for one or more cycles and (b) on day 1 of a 4 week cycle for one more cycles, wherein in each case the anti-CD39 antibody is administered at a fixed dose of 3000 mg (or optionally 2250 mg) and optionally further wherein the anti-PD(L)1 antibody is durvalumab and is administered at a fixed dose of 1500 mg.

In one embodiment, provided is a method of treating cancer or preventing recurrence of cancer, comprising administering an anti-CD39 antibody and an anti-PD(L)1 antibody, wherein the anti-CD39 antibody and the anti-PD(L)1 antibody are administered as a neoadjuvant therapy and optionally further as an adjuvant therapy, wherein the neoadjuvant therapy comprises administering anti-CD39 antibody and the anti-PD(L)1 antibody on day 1 of a 3 week cycle for one or more cycles and wherein the adjuvant therapy comprises administering anti-CD39 antibody and the anti-PD(L)1 antibody on day 1 of a 4 week cycle for one more cycles, wherein in each case the anti-CD39 antibody is administered at a fixed dose of 3000 mg (or optionally 2250 mg) and optionally further wherein the anti-PD(L)1 antibody is durvalumab and is administered at a fixed dose of 1500 mg. In one embodiment, the neoadjuvant therapy comprises 4 cycles. In one embodiment, the neoadjuvant therapy comprises at least 2,4, 8, or 12 cycles, and/or up to 12 cycles.

These aspects are more fully described in, and additional aspects, features, and advantages will be apparent from, the description provided herein.

### BRIEF DESCRIPTION OF THE DRAWINGS

FIG. 1 shows CD39 staining by immunohistochemistry on 50 squamous cell NSCLC (sqNSCLC) and 50 adenocarcinoma NSCLC (adNSCLC) FFPE samples. CD39 expression scoring was based on staining frequency and intensity. Staining is shown for stromal expression, immune cell expression and total expression. No or poor CD39 staining was observed on tumor cells. Panel A shows CD39 total score is the sum of the stromal, immune and tumor expression scores (0-60). Panel B shows Stromal score is the sum of the vascular (0-12) and connective tissue (0-12) expression scores. Panel C shows Immune score as the sum of the small immune cell (0-12) and large immune cell (0-12) scores. In each case, expression scores are shown by cancer stages (Stage I, II or III).

FIG. 2 shows ATP release from squamous NSCLC tumor cells, when the tumor cells were incubated separately with different chemotherapies.

FIG. 3A shows extracellular ATP (eATP) release by H1703 (CD39−) cells incubated with recombinant huCD39 to mimic soluble CD39 in the tumor microenvironment, with or without anti-CD39 antibody (IPH5201) and then treated with docetaxel. Docetaxel induced strong release of eATP, which was reduced in the presence of added recombinant human CD39 protein, and then restored in the presence of added IPH5201 antibody.

FIG. 3B shows eATP release in OAW42 (CD39+) cells incubated with anti-CD39 antibody (IPH5201) and treated with a dose range of docetaxel. Docetaxel induced at best a modest release of eATP at the highest concentrations in these CD39-expressing cells, however in the presence of 10 or 50 μg/mL of IPH5201, eATP accumulated significantly.

FIG. 4, left hand and right hand panel, respectively show percentage of MCA205 tumor cells expressing CD39 and quantification of adenosine, after MCA205 tumors were engrafted in human CD39 knock-in mice, treated with anti-CD39 antibody (molPH5201) or isotype control, and then harvested for analysis of CD39 expression. A significant percentage of cells express CD39 (left hand panel), and compared to control, molPH5201-treated animals showed less intratumoral adenosine (left hand panel).

FIG. 5 shows an experiment evaluating tumor growth of MC38 tumor in a cohort of huCD39 knock-in mice following treatment with gemcitabine+/−anti-CD39 antibody (molPH5201).

FIGS. 6, 7 and 8 each show an experiment evaluating tumor growth of MC38 tumor in a cohort of huCD39 knock-in mice following treatment with gemcitabine+/−anti-PD-L1⁺/−anti-CD39 antibody (molPH5201).

FIG. 9 shows a pooled analysis of three series of experiments evaluating tumor growth of MC38 tumor in huCD39 knock-in mice following treatment with gemcitabine+/−anti-PD-L1+/−anti-CD39 antibody (molPH5201).

FIG. 10 shows the indirect response PD model (inhibition on Kin) built to describe the relationship between IPH5201 concentrations and free mCD39 on monocytes.

FIG. 11A shows that anti-CD39 antibody (IPH5201) saturated binding of soluble CD39 at ≥300 mg in human patients. Each line represents data from one patient. Increase in total soluble CD39 (not shown) is consistent with increased stabilization of soluble CD39 by antibody binding. Similar trends were observed in combination treatment with durvalumab.

FIG. 11B shows that IPH5201 saturated binding of membrane-bound CD39 on immune cells from samples from human patients treated with anti-CD39 antibody (IPH5201) at 3000 mg.

FIG. 12 shows the pharmacokinetics (PK) of IPH5201 in human patients either as monotherapy (left hand panel) or as combination therapy with durvalumab (right hand panel), with the time after first dose in days on the x-axis and serum IPH5201 concentration on the Y-axis. Curves from bottom to top represent 100 mg, 300 mg, 1000 mg and 3000 mg fixed doses of IPH5201. The PK of IPH5201 was non-linear at ≤300 mg and linear at ≥1000 mg.

FIG. 13 shows a schematic of the treatment regimen for a human clinical trial. As neoadjuvant therapy, IPH5201 and durvalumab are administered at Q3W for 4 cycles at a fixed dose of 3000 mg IPH5201 and a fixed dose of 1500 mg durvalumab, in combination with chemotherapy (CT). As adjuvant therapy, IPH5201 and durvalumab are administered at Q4W for up to 12 cycles at a fixed dose of 3000 mg IPH5201 and a fixed dose of 1500 mg durvalumab.

### DETAILED DESCRIPTION OF THE INVENTION

### Definitions

Where “comprising” is used, this can optionally be replaced by “consisting essentially of” or by “consisting of”.

Human CD39, also known as “vascular” CD39, NTPdase1, ENTPD1, ATPDase and vascular ATP diphosphohydrolase, exhibits ATPase activity. CD39 hydrolyzes extracellular ATP and ADP to AMP, which is further converted to adenosine by another enzyme, 5-prime nucleotidase. The amino acid sequence of the “vascular” human CD39 mature polypeptide chain is shown in Genbank under accession number P49961, the entire disclosure of which is incorporated herein by reference, and as follows:

(SEQ ID NO: 1)
MEDTKESNVK TFCSKNILAI LGESSIIAVI
ALLAVGLTQN KALPENVKYG IVLDAGSSHT
SLYIYKWPAE KENDTGVVHQ VEECRVKGPG
ISKFVQKVNE IGIYLTDCME RAREVIPRSQ
HQETPVYLGA TAGMRLLRME SEELADRVLD
VVERSLSNYP FDFQGARIIT GQEEGAYGWI
TINYLLGKFS QKTRWESIVP YETNNQETFG
ALDLGGASTQ VTFVPQNQTI ESPDNALQER
LYGKDYNVYT HSFLCYGKDQ ALWQKLAKDI
QVASNEILRD PCFHPGYKKV VNVSDLYKTP
CTKRFEMTLP FQQFEIQGIG NYQQCHQSIL
ELENTSYCPY SQCAFNGIFL PPLQGDEGAF
SAFYFVMKEL NLTSEKVSQE KVTEMMKKFC
AQPWEEIKTS YAGVKEKYLS EYCESGTYIL
SLLLQGYHFT ADSWEHIHFI GKIQGSDAGW
TLGYMLNLIN MIPAEQPLST PLSHSTYVEL
MVLESLVLFT VAIIGLLIFH KPSYFWKDMV.

In the context herein, “neutralize” or neutralizing” when referring to the CD39 polypeptide (e.g., “neutralize CD39”, “neutralize the activity of CD39” or “neutralize the enzymatic activity of CD39”), refers to a process in which the ATP hydrolysis (ATPase) activity of CD39 is inhibited. This comprises, notably the inhibition of CD39-mediated generation of AMP and/or ADP, i.e. the inhibition of CD39-mediated catabolism of ATP to AMP and/or ADP. For membrane-bound CD39, this can be measured for example in a cellular assay that measures the capacity of a test compound to inhibit the conversion of ATP to AMP and/or ADP, either directly or indirectly. For soluble CD39, this can be measured by incubating recombinant soluble CD39 as described herein with a test compound and measuring the conversion of ATP to AMP and/or ADP, either directly or indirectly. For example, disappearance of ATP and/or generation of AMP can be assessed, as described herein, e.g., by quantifying luminescence units which are proportional to the amount of ATP present. In one embodiment, an antibody preparation causes at least a 60% decrease in the conversion of ATP to AMP, at least a 70% decrease in the conversion of ATP to AMP, or at least an 80% or 90% decrease in the conversion of ATP to AMP, referring, for example, to the assays described herein (e.g., disappearance of ATP and/or generation of AMP).

Whenever “treatment of cancer” or the like is mentioned with reference to anti-CD39 binding agent (e.g., antibody), this can include: (a) method of treatment of cancer, said method comprising the step of administering (for at least one treatment) an anti-CD39 binding agent, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-CD39 binding agent for the treatment of cancer, or an anti-CD39 binding agent, for use in said treatment (especially in a human); (c) the use of an anti-CD39 binding agent for the manufacture of a pharmaceutical preparation for the treatment of cancer, a method of using an anti-CD39 binding agent for the manufacture of a pharmaceutical preparation for the treatment of cancer, optionally comprising admixing an anti-CD39 binding agent with a pharmaceutically acceptable carrier, or a pharmaceutical preparation comprising an effective dose of an anti-CD39 binding agent that is appropriate for the treatment of cancer; or (d) any combination of a), b), and c), in accordance with the subject matter allowable for patenting in a country where this application is filed.

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “antibody,” as used herein, can include polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., CD39, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody (e.g. antibody having the amino acid sequences of SEQ ID NOS: 2-7, 8-9 or 10-11), it means that the antibody competes with the monoclonal antibody in a binding assay using either recombinant CD39 molecules or surface expressed CD39 molecules. For example, if a test antibody reduces the binding of a reference antibody to a CD39 polypeptide or CD39-expressing cell in a binding assay, the antibody is said to “compete” respectively with the reference antibody.

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc, and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein, a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term “conformational” is therefore often used interchangeably with “structural”.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein (e.g. antibody or antibody fragment), or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### Treatment of Cancer

The disclosure generally relates to methods for treating cancer in a patient using an anti-CD39 antibody in combination with an anti-PD(L)1 antibody as neoadjuvant and optionally further as adjuvant therapy. The treatments can thus be useful as preoperative therapy and optionally further as postoperative therapy in patients having a cancer or tumor that is resectable or that can potentially or likely become resectable. Optionally the use as preoperative therapy is in further in combination with postoperative chemotherapy. The methods are particularly advantageous in the treatment of lung cancer, particularly non-small cell lung cancer (NSCLC). In one embodiment, the cancer is a stage II or IIIA NSCLC, e.g. a resectable stage II or IIIA NSCLC. The methods are thus particularly advantageous in the treatment of tumor or cancers where surgery is the main treatment.

In one embodiment, disclosed is a method of reducing or inhibiting tumor growth in a subject in need thereof, comprising administering to the subject a therapeutically effective amount of each of anti-CD39 antibody, an anti-PD(L)1 antibody and optionally a chemotherapy agent. In one embodiment, disclosed a method of treating cancer, in particular a stage II or IIIA NSCLC, in a subject in need thereof, comprising administering to the subject a therapeutically effective amount of each of anti-CD39 antibody, an anti-PD(L)1 antibody and optionally a chemotherapy agent. In one embodiment the anti-CD39 antibody, an anti-PD(L)1 antibody and optionally a chemotherapy agent are administered as preoperative therapy. In one embodiment the anti-CD39 antibody and an anti-PD(L)1 antibody are administered as preoperative therapy and as postoperative therapy.

The methods and regimens herein can be useful for example for treating and/or preventing the recurrence of a tumor or cancer in a patient, for preventing metastases or the recurrence of metastases, for preventing the progression or growth of a tumor or cancer, for preventing the progression or growth of a tumor or cancer, and/or for improving or increasing survival, time to recurrence (e.g. time-to-distant recurrence), and/or recurrence-free survival. The patient can optionally be specified as having a surgically resectable cancer or tumor. The methods and regimens herein can also be useful for making a tumour more operable, and/or improving the likelihood of a complete resection. Optionally, the methods herein are characterized as a method of improving the likelihood of being free of residual disease (e.g. minimum residual disease MRD) and/or being ctDNA-negative (not having detectable ctDNA), e.g., after surgical resection. The methods and regimens herein can also be useful for example for increasing or potentiating an anti-tumor immune response, inhibiting the enzymatic activity of CD39 (e.g. in a tumor), increasing the intratumoral concentration of ATP, decreasing the intratumoral concentration of adenosine, increasing the activity and/or activity of T cells, NK cells, tumor infiltrating NK cells, tumor infiltrating T cells and/or dendritic cells.

The anti-CD39 antibodies used herein are antibodies that neutralize CD39. “Neutralize” or neutralizing”, when referring to the CD39 polypeptide (e.g., “neutralize CD39”, “neutralize the activity of CD39” or “neutralize the enzymatic activity of CD39”), refers to a process in which the ATP hydrolysis (ATPase) activity of CD39 is inhibited. Such antibodies have been reported in several publications, supra, the disclosures of amino acid sequences of which are incorporated herein by reference. Some antibodies may neutralize membrane-bound CD39 by inhibiting the domain motion of membrane-bound CD39 (memCD39), however without similarly affecting the activity of the soluble CD39 protein (sCD39). It has been reported that memCD39 occurs as a homo-multimer while sCD39 is a monomer, and moreover that the transmembrane domains in memCD39 undergo dynamic motions that underlie a functional relationship with the active site. Consequently, unlike sCD39, memCD39 may present a setting that makes antibody-mediated neutralization possible. One possibility is that use of a bivalent antibody that binds simultaneously to two memCD39 molecules (e.g., within a memCD39 homo-multimer) is required for functional neutralization.

The anti-CD39 antibodies described herein bind an epitope present on human CD39 protein expressed at the surface of cells (e.g. they can compete with the anti-CD39 antibody of SEQ ID NOS: 10 and 11 for binding to an epitope on CD39), including tumor cells and potently inhibit the enzymatic (ATPase activity) activity of the cell membrane bound CD39 enzyme (CD39 as expressed at the surface of cells), and the anti-CD39 antibodies described herein further inhibit the enzymatic (ATPase activity) activity of soluble (extracellular domain) human CD39 protein. The antibodies thereby mediate strong neutralization of CD39 activity in an individual by neutralizing both membrane-bound and soluble CD39 protein, including soluble CD39 released or shed from tumor cells, thereby reducing immunosuppression, e.g., for the treatment of cancer and/or infectious disease.

In one aspect, the anti-CD39 antibody comprises (a) a HCDR1, HCDR2 and HCDR3 of SEQ ID NOS: 2, 3 and 4, respectively, and (b) an LCDR1, LCDR2 and LCDR3 sequence of SEQ ID NOS: 5, 6 and 7, respectively.

In one aspect, the anti-CD39 antibody can be characterized as comprising: a HCDR1 comprising an amino acid sequence: DYNMH (SEQ ID NO: 2), or a sequence of at least 3 or 4 contiguous amino acids thereof, optionally wherein one or more of these amino acids may be substituted by a different amino acid; a HCDR2 comprising an amino acid sequence: YIVPLNGGSTFNQKFKG (SEQ ID NO: 3), or a sequence of at least 4, 5, 6, 7, 8, 9 or 10 contiguous amino acids thereof, optionally wherein one or more of these amino acids may be substituted by a different amino acid, optionally wherein the asparagine at Kabat position 61 is substituted, optionally wherein the lysine at Kabat position 65 is substituted; a HCDR3 comprising an amino acid sequence: GGTRFAY (SEQ ID NO: 4), or a sequence of at least 4, 5 or 6 contiguous amino acids thereof, optionally wherein one or more of these amino acids may be substituted by a different amino acid; a LCDR1 comprising an amino acid sequence: RASESVDNFGVSFMY (SEQ ID NO: 5), or a sequence of at least 4, 5, 6, 7, 8, 9 or 10 contiguous amino acids thereof, optionally wherein one or more of these amino acids may be substituted by a different amino acid, optionally wherein the arginine at Kabat position 24 is substituted; a LCDR2 region comprising an amino acid sequence: GASNQGS (SEQ ID NO: 6) or a sequence of at least 4, 5 or 6 contiguous amino acids thereof, optionally wherein one or more of these amino acids may be substituted by a different amino acid; and/or a LCDR3 region comprising an amino acid sequence: QQTKEVPYT (SEQ ID NO: 7), or a sequence of at least 4, 5, 6, 7 or 8 contiguous amino acids thereof, optionally wherein one or more of these amino acids may be deleted or substituted by a different amino acid.

In any embodiment, CDR positions may be according to Kabat numbering.

In one aspect, the anti-CD39 antibody comprises the hypervariable region of, optionally the CDRs of, the antibody having the VH and VL of SEQ ID NOS: 8 and 9, shown below.

### Anti-CD39 VH:

(SEQ ID NO: 8)
QVQLVQSGAEVKKPGASVKVSCKASGYTFTDYNMHWVRQAPGQRL
EWIGYIVPLNGGSTFNQKFKGRATITVDTSARTAYMELSSLRSED
TAVYYCARGGTRFAYWGQGTLVTVSS.

### Anti-CD39 VL:

(SEQ ID NO: 9)
DIVMTQSPDSLAVSLGERATINCRASESVDNFGVSFMYWFQQKPG
QPPKLLIYGASNQGSGVPDRFSGSGSGTDFTLTISSLQAEDVAVY
YCQQTKEVPYTFGGGTKVEIK.

In one embodiment, the anti-CD39 antibody can be characterized by an antigen binding domain comprising a heavy chain variable region (VH) comprising a CDR1, CDR2 and CDR3 having the respective amino acid sequences shown in SEQ ID NOS: 2, 3 and 4 and framework FR1, FR2 and FR3 amino acid sequences derived from the human IGHV1-3 gene, e.g., IGHV1-3*01 (and optionally further framework 4 (FR4) amino acid sequences derived from the human IGHJ1 gene, e.g. IGHJ1*01); and a light chain variable region (VL) CDR1, CDR2 and CDR3 having the respective amino acid sequences shown in SEQ ID NOS: 5, 6 and 7, and framework FR1, FR2 and FR3 amino acid sequences derived from the human IGKV4-1 (e.g. IGK4-1*01) gene, and optionally further framework 4 (FR4) amino acid sequences derived from the human IGKJ4 (e.g. IGKJ4*01) gene. The VH further comprises one or more amino acid substitutions of a residue present in a human framework sequence by a different residue (e.g. a residue present in a non-human framework) at Kabat positions selected from the group consisting of 48, 67, 71 and 76. In one embodiment, the VH comprises one or more amino acid substitutions in the heavy chain CDR2, e.g. at Kabat positions 60 and/or 64. Optionally the residue at position 60 is a serine (e.g. the CDR2 comprises a N60S substitution). Optionally the residue present at Kabat position 64 is a glutamine (e.g., the CDR2 comprises a K64Q substitution). In one embodiment, the residue present in the VL at Kabat position 24 is a lysine (e.g. the CDR1 comprises a R24K substitution). Optionally wherein a phenylalanine is present in the VL at Kabat position 36.

In one embodiment, a VH comprises an isoleucine residue at Kabat position 48, an alanine residue at Kabat position 67, a valine at Kabat position 71 and an arginine at Kabat position 76.

In one embodiment, a VL comprises a phenylalanine at Kabat position 36 (FR2). In one embodiment, a VL comprises a lysine at Kabat position 24 (CDR1).

In any embodiment, an anti-CD39 antibody can be characterized as comprising a heavy chain variable region (VH) comprising an amino acid sequence at least 70%, 80%, 85%, 90%, 92%, 94%, 95%, 96%, 97%, 98% or 99% identical to the amino acid sequence of SEQ ID NO: 8, and a light chain variable region (VL) comprising an amino acid sequence at least 70%, 80%, 85%, 90%, 92%, 94%, 95%, 96%, 97%, 98% or 99% identical to the amino acid sequence of SEQ ID NO: 9.

In one embodiment, an anti-CD39 antibody comprises a heavy chain variable region (VH) comprising CDR1, CDR2 and CDR3 having the respective amino acid sequences shown in SEQ ID NOS: 2, 3 and 4 and human frameworks (e.g., FR1, FR2, FR3 and FR4 of human origin); and a light chain variable region (VL) CDR1, CDR2 and CDR3 comprising the respective amino acid sequences shown in SEQ ID NOS: 5, 6 and 7, and human frameworks (e.g., FR1, FR2, FR3 and FR4 of human origin), wherein the (VH) comprises an amino acid sequence at least 70%, 80%, 85%, 90%, 92%, 94%, 95%, 96%, 97%, 98% or 99% identical to the amino acid sequence of SEQ ID NOS: 8, and a light chain variable region (VL) comprising an amino acid sequence at least 70%, 80%, 85%, 90%, 92%, 94%, 95%, 96%, 97%, 98% or 99% identical to the amino acid sequence of SEQ ID NO: 9.

In any embodiment, a VH can be characterized as comprising a substitution at one, two, three or all of the Kabat positions 48, 67, 71 and 76. In one embodiment, the residue at position 48 is an isoleucine (e.g., a M48I substitution). In one embodiment, the residue at position 67 is an alanine (e.g., a V67A substitution). In one embodiment, the residue at position 71 is a valine (e.g., a R71V substitution). In one embodiment, the residue at position 76 is an arginine (e.g., a S76R substitution). In any embodiment, a VL can be characterized as comprising a substitution at Kabat position 36. In one embodiment, the residue at position 36 is a phenylalanine (e.g., a Y36F substitution).

In one embodiment, an anti-CD39 antibody comprises a heavy chain comprising an amino acid sequence at least 70%, 80%, 85%, 90%, 92%, 94%, 95%, 96%, 97%, 98% or 99% identical to the amino acid sequence of SEQ ID NO: 10, and a light chain comprising an amino acid sequence at least 70%, 80%, 85%, 90%, 92%, 94%, 95%, 96%, 97%, 98% or 99% identical to the amino acid sequence of SEQ ID NO: 11.

Anti-CD39 Heavy chain
(SEQ ID NO: 10)
QVQLVQSGAEVKKPGASVKVSCKASGYTFTDYNMHWVRQAPGQRL
EWIGYIVPLNGGSTFNQKFKGRATITVDTSARTAYMELSSLRSED
TAVYYCARGGTRFAYWGQGTLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKRVEPKSCDKTHTC
PPCPAPEAEGAPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPSSIEKTISKAKGQPREPQVYTLPPSREEMTKN
QVSLTCLVKGFYPSDIAVEWESNGQPENNYKTTPPVLDSDGSFFL
YSKLTVDKSRWQQGNVFSCSVMHEALHNHYTQKSLSLSPGK.
Anti-CD39 Light chain
(SEQ ID NO: 11)
DIVMTQSPDSLAVSLGERATINCRASESVDNFGVSFMYWFQQKPG
QPPKLLIYGASNQGSGVPDRFSGSGSGTDFTLTISSLQAEDVAVY
YCQQTKEVPYTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC.

In one embodiment, an anti-CD39 antibody comprises a heavy chain comprising the amino acid sequence of SEQ ID NO: 10 and a light chain comprising the amino acid sequence of SEQ ID NO: 11. In one embodiment, the anti-CD39 antibody is IPH5201.

In any embodiment, an anti-CD39 antibody can be characterized as binding to and inhibiting or neutralizing the ATPase activity of a soluble CD39 protein (sCD39). In one embodiment the sCD39 protein lacks the two transmembrane domains (i.e. the transmembrane domains near the N- and C-terminal ends) found in membrane bound CD39. In one embodiment, sCD39 is a non-membrane bound sCD39 protein found in circulation, e.g., in a human individual. In one embodiment, the CD39-derived sequence of a sCD39 protein comprises or consists of the Thr38-Val478 fragment of CD39. Thr38-Val478 protein with C-terminal His tag is available commercially from R&D Systems, Inc., (product number 4397-EN). In one embodiment, the protein, antibody or antibody fragment inhibits the ATPase activity of SCD39 when incubated with sCD39 in solution, e.g. in tumor cell supernatants. In one embodiment, the protein, antibody or antibody fragment specifically binds the human CD39 protein, both in soluble (extracellular domain protein) and in membrane-bound form.

The anti-CD39 antibody described herein comprises a human Fc domain that is modified to have decreased or substantially lack binding to a human Fcγ receptor, e.g., one or more (or all of) human CD16, CD32a, CD32b and CD64. The antibody does not depend on ADCC-, CDC- or toxin-mediated depletion of CD39-expressing cells for their CD39 inhibitory activity. These antibody is thus used as “pure” CD39 blockers, with an immunomodulatory activity.

As used herein, the terms “PD-1” refers to the protein Programmed Death 1 (PD-1) (also referred to as “Programmed Cell Death 1”), an inhibitory member of the CD28 family of receptors, that also includes CD28, CTLA-4, ICOS and BTLA. The complete human PD-1 sequence can be found under GenBank Accession No. U64863, shown as follows:

(SEQ ID NO: 12)
MQIPQAPWPVVWAVLQLGWRPGWFLDSPDRPWNPPTFFPALLVVT
EGDNATFTCSFSNTSESFVLNWYRMSPSNQTDKLAAFPEDRSQPG
QDCRFRVTQLPNGRDFHMSVVRARRNDSGTYLCGAISLAPKAQIK
ESLRAELRVTERRAEVPTAHPSPSPRPAGQFQTLVVGVVGGLLGS
LVLLVWVLAVICSRAARGTIGARRTGQPLKEDPSAVPVFSVDYGE
LDFQWREKTPEPPVPCVPEQTEYATIVFPSGMGTSSPARRGSADG
PRSAQPLRPEDGHCSWPL.

“PD-1” also includes any variant, derivative, or isoform of the PD-1 gene or encoded protein. PD-1 is expressed on activated B cells, T cells, and myeloid cells (Okazaki et al. (2002) Curr. Opin. Immunol. 14:391779-82; Bennett et al. (2003) J Immunol 170:711-8). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members.

The complete human PD-L1 sequence can be found under UniProtKB/Swiss-Prot, identifier Q9NZQ7-1, shown as follows:

(SEQ ID NO: 13)
MRIFAVFIFMTYWHLLNAFTVTVPKDLYVVEYGSNMTIECKFPVE
KQLDLAALIVYWEMEDKNIIQFVHGEEDLKVQHSSYRQRARLLKD
QLSLGNAALQITDVKLQDAGVYRCMISYGGADYKRITVKVNAPYN
KINQRILVVDPVTSEHELTCQAEGYPKAEVIWTSSDHQVLSGKTT
TTNSKREEKLFNVTSTLRINTTTNEIFYCTFRRLDPEENHTAELV
IPELPLAHPPNERTHLVILGAILLCLGVALTFIFRLRKGRMMDVK
KCGIQDTNSKKQSDTHLEET.

PD-L1 is abundant in a variety of human cancers. The interaction between PD-1 and PD-L1 results in a decrease in tumor infiltrating lymphocytes, a decrease in T-cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

An anti-PD(L)1 antibody is an antibody that binds to PD-1 or PD-L1. The antibody can neutralize PD-1 or reduce the inhibitory activity of human PD-1. “Reduces the inhibitory activity of human PD-1”, “neutralizes PD-1” or “neutralizes the inhibitory activity of human PD-1” refers to a process in which PD-1 is inhibited in its signal transduction capacity resulting from the interaction of PD-1 with one or more of its binding partners, such as PD-L1 or PD-L2. An antibody that neutralizes the inhibitory activity of PD-1 decreases, blocks, inhibits, abrogates or interferes with signal transduction resulting from the interaction of PD-1 with one or more of its binding partners, in particular as PD-L1. Such an agent can thereby reduce the negative co-stimulatory signal mediated by or through cell surface proteins expressed on T lymphocytes, so as to enhance T-cell effector functions such as proliferation, cytokine production and/or cytotoxicity. A PD-1 neutralizing agent can interact with PD-1 and/or with one or more of its binding partners, e.g. PD-L1 and PD-L2.

In some embodiments, the anti-PD(L)1 antibody is an anti-PD-L1 monoclonal antibody that inhibits the binding of PD-L1 to PD-1. In some embodiments, the anti-PD(L)1 antibody is an anti-PD-1 monoclonal antibody that inhibits the binding of PD-1 to PD-L1.

In some embodiments, the anti-PD(L)1 antibody is YW243.55.S70, MPDL3280A (atezolizumab, Tecentriq®), MDX-1105, or durvalumab (MEDI4736, Imfinzi®). MDX-1105, also known as BMS-936559, is an anti-PD-L1 antibody described in WO2007/005874. Antibody YW243.55.S70 is an anti-PD-L1 described in WO 2010/077634. Examples of anti-PD-L1 antibodies useful for the methods disclosed herein, and methods for making thereof are also described in WO 2010/077634 A1 and U.S. Pat. No. 8,217,149, which are incorporated herein by reference.

In some embodiments, the anti-PD(L)1 antibody is a PD-L1 antibody that is durvalumab. Durvalumab (MEDI4736, Imfinzi™) is a human monoclonal antibody directed against human PD-L1 that is capable of blocking the binding of PD-L1 to both the PD-1 and CD80 receptors. Disclosure related to durvalumab can be found in U.S. Pat. Nos. 8,779,108 and 9,493,565, which are incorporated herein by reference. Durvalumab has the heavy and light chains of amino acid sequences SEQ ID NO: 16 and SEQ ID NO: 17, respectively. The heavy chain variable region of durvalumab is shown in SEQ ID NO: 14 and the light chain variable region of durvalumab is shown in SEQ ID NO: 15.

In another embodiment, the anti-PD(L)1 antibody is an anti-PD-L1 antibody (or an antigen-binding portion thereof) competing with durvalumab for binding to PD-L1. In some embodiments, the anti-PD-L1 antibody binds to the same epitope as durvalumab. In certain embodiments, the anti-PD-L1 antibody has the same heavy and light chain CDRs as durvalumab.

In some embodiments, the anti-PD(L)1 antibody (e.g. an agent derived from durvalumab) comprises (i) the heavy chain variable region of SEQ ID NO: 14, or an amino acid sequence at least 50%, 60%, 70%, 80%, 90%, 95%, 98% or 99% identical thereto, and (ii) the light chain variable region of SEQ ID NO: 15, or an amino acid sequence at least 50%, 60%, 70%, 80%, 90%, 95%, 98% or 99% identical thereto. In some embodiments, the PD-1 neutralizing agent (e.g. an agent derived from durvalumab) comprises (i) the heavy chain of SEQ ID NO: 16, or an amino acid sequence at least 50%, 60%, 70%, 80%, 90%, 95%, 98% or 99% identical thereto, and (ii) the light chain of SEQ ID NO: 17, or an amino acid sequence at least 50%, 60%, 70%, 80%, 90%, 95%, 98% or 99% identical thereto. In some embodiments, the PD-1 neutralizing agent comprises H-CDR1, H-CDR2 and/or H-CDR3 sequences derived from the heavy chain variable region comprising the amino acid sequence of SEQ ID NO: 14. In some embodiments, the PD-1 neutralizing agent comprises L-CDR1, L-CDR2 and/or L-CDR3 sequences derived from the light chain variable region comprising the amino acid sequence of SEQ ID NO: 15.

In some embodiments, the anti-PD(L)1 antibody comprises the heavy chain H-CDR1, H-CDR2 and H-CDR3 domains having the amino acid sequences of SEQ ID NOS: 18-20, respectively, and the light chain L-CDR1, L-CDR2, L-CDR3 domains having the amino acid sequences of SEQ ID NOS: 21-23, respectively.

### Heavy Chain Variable Region of Durvalumab:

(SEQ ID NO: 14)
EVQLVESGGGLVQPGGSLRLSCAASGFTFSRYWMSWVRQAPGKGL
EWVANIKQDGSEKYYVDSVKGRFTISRDNAKNSLYLQMNSLRAED
TAVYYCAREGGWFGELAFDYWGQGTLVTVSS.

### Light Chain Variable Region of Durvalumab:

(SEQ ID NO: 15)
EIVLTQSPGTLSLSPGERATLSCRASQRVSSSYLAWYQQKPGQAP
RLLIYDASSRATGIPDRFSGSGSGTDFTLTISRLEPEDFAVYYCQ
QYGSLPWTFGQGTKVEIK.

### Heavy Chain of Durvalumab:

(SEQ ID NO: 16)
EVQLVESGGGLVQPGGSLRLSCAASGFTFSRYWMSWVRQAPGKGL
EWVANIKQDGSEKYYVDSVKGRFTISRDNAKNSLYLQMNSLRAED
TAVYYCAREGGWFGELAFDYWGQGTLVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKRVEPKSCD
KTHTCPPCPAPEFEGGPSVFLFPPKPKDTLMISRTPEVTCVVVDV
SHEDPEVKFNWYVDGVEVHNAKTKPREEQYNSTYRVVSVLTVLHQ
DWLNGKEYKCKVSNKALPASIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVMHEALHNHYTQKSLSLSPG
K.

### Light Chain of Durvalumab:

(SEQ ID NO: 17)
EIVLTQSPGTLSLSPGERATLSCRASQRVSSSYLAWYQQKPGQAP
RLLIYDASSRATGIPDRFSGSGSGTDFTLTISRLEPEDFAVYYCQ
QYGSLPWTFGQGTKVEIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC.

### Durvalumab, Heavy Chain CDRs:

H-CDR1:
(SEQ ID NO: 18)
GFTFSRYWMS
H-CDR2:
(SEQ ID NO: 19)
NIKQDGSEKYYVDSVKG
H-CDR3:
SEQ ID NO: 20)
EGGWFGELAFDY

### Durvalumab, Light Chain CDRs:

L-CDR1:
(SEQ ID NO: 21)
RASQRVSSSYLA
L-CDR2:
(SEQ ID NO: 22)
DASSRAT
L-CDR3:
(SEQ ID NO: 23)
QQYGSLPWT

In another embodiment, the anti-PD(L)1 antibody is atezolizumab (MPDL3280A, Tecentriq®, CAS Registry Number: 1422185-06-5). In some embodiments, the anti-PD-L1 antibody comprises a heavy chain variable region comprising the amino acid sequence:

(SEQ ID NO: 24)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSS
or
(SEQ ID NO: 25)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTK

and a light chain variable region comprising the amino acid sequence:

(SEQ ID NO: 26)
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKR.

In some embodiments, anti-PD(L)1 antibody comprises (i) a heavy chain or heavy chain variable region of SEQ ID NO: 27, or an amino acid sequence at least 50%, 60%, 70%, 80%, 90%, 95%, 98% or 99% identical thereto, and (ii) a light chain or light chain variable region of SEQ ID NO: 28, or an amino acid sequence at least 50%, 60%, 70%, 80%, 90%, 95%, 98% or 99% identical thereto.

(SEQ ID NO: 28)
EVQLVESGGGLVQPGGSLRLSCAASGFTFSDSWIHWVRQAPGKGL
EWVAWISPYGGSTYYADSVKGRFTISADTSKNTAYLQMNSLRAED
TAVYYCARRHWPGGFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYASTYRVVSVLTVLHQDWL
NGKEYKCKVSNKALPAPIEKTISKAKGQPREPQVYTLPPSREEMT
KQVSLTCLVKGFYPSDIAVEWESNGQPENYKTTPPVLDSDGSFFL
YSKLTVDKSRWQQGNVFSCSVMHEALHNHYTQKSLSLSPG.
(SEQ ID NO: 27)
DIQMTQSPSSLSASVGDRVTITCRASQDVSTAVAWYQQKPGKAPK
LLIYSASFLYSGVPSRFSGSGSGTDFTLTISSLQPEDFATYYCQQ
YLYHPATFGQGTKVEIKRTVAAPSVFIFPPSDEQLKSGTASVVCL
LNFYPREAKVQWKVDNALQSGNSQESVTEQDSKDSTYSLSSTLTL
SKADYEKH KVYACEVTHQGLSSPVTKSFNRGEC.

In some embodiments, the anti-PD(L)1 antibody is an anti-PD-1 antibody that inhibits the binding of PD-1 to PD-L1. In some embodiments, the anti-PD-1 antibody is nivolumab. Nivolumab (also known as OPDIVOR; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., (2014) Cancer Immunol Res. 2 (9): 846-56). In another embodiment, the anti-PD-1 antibody or fragment thereof competes with nivolumab for binding to PD-1. In some embodiments, the anti-PD-1 antibody binds to the same epitope as nivolumab. In certain embodiments, the anti-PD-1 antibody has the same heavy and light chain CDRs as nivolumab.

In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1. Pembrolizumab is described, for example, in U.S. Pat. No. 8,900,587. Pembrolizumab has been approved by the FDA for the treatment of relapsed or refractory melanoma and advanced NSCLC. In another embodiment, the anti-PD-1 antibody (or an antigen-binding portion thereof) competes with pembrolizumab for binding to PD-1. In some embodiments, the anti-PD-1 antibody binds to the same epitope as pembrolizumab. In certain embodiments, the anti-PD-1 antibody has the same heavy and light chain CDRs as pembrolizumab.

In another embodiment, the anti-PD-1 antibody is cemiplimab.

A chemotherapeutic agent refers to a compound useful in the treatment of cancer, for example lung cancer, NSCLC, optionally stage II NSCLC and/or stage IIIA NSCLC. For example, a chemotherapeutic agent can be a compound known to be useful in the neoadjuvant treatment of NSCLC, optionally resectable or II or IIIA NSCLC. The chemotherapy can be used for example in a known or standard dose and frequency of administration for such chemotherapy when used as neoadjuvant therapy. See for example Burdett S., 2014, supra, for examples of neoadjuvant chemotherapy and regimens. In some embodiments, the chemotherapy used in the neoadjuvant treatment herein comprises a platinum-based chemotherapy agent. A “platinum-based” chemotherapeutic agent comprises an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. In some instances, platinum-based chemotherapeutic agents (e.g., cisplatin or carboplatin) may be administered in combination with one or more additional chemotherapeutic agents, e.g., a nucleoside analog (e.g., gemcitabine).

For example, a platinum-based chemotherapy may include a platinum-based chemotherapeutic agent (e.g., cisplatin or carboplatin), and, optionally, one or more additional chemotherapeutic agents, e.g., a nucleoside analog (e.g., gemcitabine), a taxane (e.g. paclitaxel) or a folate antimetabolite (e.g. pemetrexed).

In some embodiments, the chemotherapy used in the neoadjuvant treatment herein comprises gemcitabine.

In some embodiments, the chemotherapy used in the neoadjuvant treatment herein comprises administration of two or more chemotherapy agents. In some embodiments, the chemotherapy comprises administration of at least one or two agents selected from: paclitaxel, carboplatin, pemetrexed, or cisplatin. In some embodiments, the chemotherapy comprises or consists of administration of carboplatin and paclitaxel. In some embodiments, the chemotherapy comprises or consists of administration of cisplatin and gemcitabine. In some embodiments, the chemotherapy comprises or consists of administration of cisplatin and pemetrexed. In some embodiments, the chemotherapy comprises or consists of administration of carboplatin and pemetrexed.

As disclosed herein, the disclosure provides a method of treating a tumor or cancer and/or preventing the recurrence of a tumor or cancer in a patient in need thereof, e.g. in a patient having a resectable cancer or tumor, comprising administering an anti-CD39 antibody and an anti-PD(L)1 antibody, wherein the anti-CD39 antibody and the anti-PD(L)1 antibody are administered as neoadjuvant therapy. In one embodiment, the method comprises administering an anti-CD39 antibody, the anti-PD(L)1 antibody and a chemotherapeutic agent, wherein the anti-CD39 antibody, the anti-PD(L)1 antibody and chemotherapeutic agent are administered as neoadjuvant therapy.

The disclosure further provides a method of treating a tumor or cancer and/or preventing the recurrence of a tumor or cancer in a patient in need thereof, comprising administering an anti-CD39 antibody and an anti-PD(L)1 antibody, wherein the anti-CD39 antibody and the anti-PD(L)1 antibody are administered as a neoadjuvant therapy and then further as an adjuvant therapy.

The term “patient” is intended to include human and non-human animals, particularly mammals. In some embodiments, the disclosure relates to providing treatments for a patient having a tumor disorder and/or a cancer disorder (methods of treatment, or pharmaceutical formulations for use as described herein), optionally a surgically resectable tumor and/or cancer. In some embodiments, the tumor is a lung tumor (e.g., non-small cell lung cancer (NSCLC)). In some embodiments, the non-small cell lung tumor is a squamous cell carcinoma, an adenocarcinoma, or a large cell carcinoma. In some embodiments, the patient has a non-small cell lung tumor that is stage I, stage II, or stage III. The methods can be used as first line treatment (1L treatment, 1-L treatment, 1L or 1-L), i.e. the first treatment given for a disease, particularly a cancer as described herein. A first line treatment may be specific for a given type or subtype of cancer, or a specific cancer stage. A first line treatment may be part of a standard set of treatments. A first-line treatment is generally accepted as the best treatment for a disease, particularly a cancer as described herein. If a first line treatment does not cure the disease or it causes severe side effects, subsequent lines of treatment may be used instead. In some embodiments, the disclosure relates to providing first line treatments for cancer (methods of treatment, or pharmaceutical formulations for use as described herein).

In some embodiments, prior to receiving a neoadjuvant treatment of the disclosure the patient has not previously undergone resection of a non-small cell lung tumor. In some embodiments, prior to receiving an adjuvant treatment of the disclosure the patient has undergone a resection, optionally a complete resection, of a non-small cell lung tumor.

Provided here further are treatment regimens adapted for the administration of the anti-CD39 antibody and the anti-PD(L)1 antibody as neoadjuvant and/or adjuvant therapy. Accordingly in an administration, treatment or prevention method herein, the method can be specified as comprising administering to the patient an effective amount of a treatment regimen comprising an anti-CD39 antibody and an anti-PD(L)1 antibody, e.g., a treatment regimen described herein comprising an anti-CD39 antibody and an anti-PD(L)1 antibody. Accordingly provided herein are methods of treating a lung cancer in a patient, the method comprising administering to the patient an effective amount of a treatment regimen comprising an anti-CD39 antibody and an anti-PD(L)1 antibody, and optionally further a chemotherapy, wherein the treatment regimen is a neoadjuvant therapy. Optionally the method further comprises administering to the patient an effective amount of a treatment regimen comprising an anti-CD39 antibody and an anti-PD(L)1 antibody, wherein the treatment regimen is an adjuvant therapy. Doses and dosing intervals for use in the treatment regimens can be specified as further provided herein, including advantageous regimens in which the same doses of anti-CD39 antibody and anti-PD(L)1 antibody can be used in both the neoadjuvant setting and the adjuvant setting.

Accordingly, in one embodiment, a treatment regimen of the disclosure can be characterized as comprising administering to the patient, before surgical tumor resection, an anti-CD39 antibody and an anti-PD(L)1 antibody (and optionally further a chemotherapy). The treatment can further comprise administering to the patient, after surgical tumor resection, an anti-CD39 antibody and an anti-PD(L)1 antibody. In one embodiment, the anti-PD(L)1 antibody is durvalumab. The tumor or cancer can optionally be specified as being as a tumor or cancer that is deemed to be surgically resectable.

In any embodiment herein, a method and/or an effective amount can be specified to include an amount of a combination of anti-PD(L)1 antibody and anti-CD39 antibody (and optionally further chemotherapy) that achieves a therapeutic result. In some examples, an effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), disease-specific survival (DSS), distant metastasis-free survival, progression-free survival (PFS) and/or overall survival (OS)), improved duration of response (DOR), improved time to deterioration of function and quality of life (QoL), and/or ctDNA clearance. Improvement (e.g., in terms of response rate (e.g., ORR, CR, and/or PR), survival (e.g., DFS, DSS, distant metastasis-free survival, PFS, and/or OS), DOR, improved time to deterioration of function and QoL, undetectable ctDNA and/or ctDNA clearance) may be relative to a suitable reference, for example, observation or a reference treatment (e.g., treatment that does not include the anti-CD39-antibody (e.g., treatment with placebo, treatment with anti-PD(L)1 antibody)). In some instances, improvement is (e.g., in terms of response rate (e.g., ORR, CR, and/or PR), survival (e.g., EFS, DFS, DSS, distant metastasis-free survival, PFS, and/or OS), DOR, improved time to deterioration of function and QoL, and/or ctDNA clearance or undetectable ctDNA status) may be relative to observation.

As used herein, “complete response” and “CR” refers to disappearance of all target lesions.

As used herein, “partial response” and “PR” refers to at least a defined (e.g. 30%) decrease in the sum of the longest diameters (SLD) of target lesions, taking as reference the baseline SLD prior to treatment.

As used herein, “overall response rate,” “objective response rate,” and “ORR” refer interchangeably to the sum of CR rate and PR rate.

As used herein, “disease-free survival” and “DFS” refer to the length of time after a primary treatment (e.g., surgical resection) that the patient survives without recurrence of the cancer.

As used herein, “disease-specific survival” and “DSS” refer to the length of time that the patient has not died from a specific disease (e.g., NSCLC). In some instances, DSS may be defined as the time from randomization to death from NSCLC (e.g., per investigator assessment of cause of death). As used herein, “distant metastasis-free survival” refers to the length of time from either the date of diagnosis or the start of treatment that a patient is still alive and the cancer has not spread to other parts of the body. In some instances, distant metastasis-free survival is defined as the time from randomization to the diagnosis of distant (i.e., non-locoregional) metastases or death from any cause.

As used herein, “progression-free survival” and “PFS” refer to the length of time during and after treatment during which the cancer does not get worse. PFS may include the amount of time patients have experienced a CR or a PR, as well as the amount of time patients have experienced stable disease.

As used herein, “overall survival” and “OS” refer to the length of time from either the date of diagnosis or the start of treatment for a disease (e.g., cancer) that the patient is still alive. For example, OS may be defined as the time from randomization to death from any cause.

As used herein, the term “duration of response” and “DOR” refer to a length of time from documentation of a tumor response until disease progression or death from any cause, whichever occurs first.

In one embodiment, the treatment regimens herein permit administration of the anti-CD39 antibody at the same dose (e.g. 2250 mg or 3000 mg fixed dose of the anti-CD39 antibody) in both the adjuvant setting and in the neoadjuvant setting. Furthermore, the regimens permit the anti-CD39 antibody to be administered every three weeks as neoadjuvant and every four weeks as adjuvant (at the same dosage in both neoadjuvant and adjuvant therapy).

In one embodiment, provided is a method for administering an anti-CD39 antibody (e.g. an anti-CD39 antibody having the characteristics described herein; an antibody comprising the amino acid sequences of SEQ ID NOS: 2-7, SEQ ID NOS: 8 and 9 or SEQ ID NOS: 10 and 11), wherein the antibody is administered Q3w and/or Q4w at a fixed dose of 3000 mg. In one embodiment, the anti-CD39 antibody is administered Q3w at a fixed dose of 3000 mg as neoadjuvant therapy and Q4w at a dose of 3000 mg as adjuvant therapy. In another embodiment, provided is a method for administering an anti-CD39 antibody (e.g. an anti-CD39 antibody having the characteristics described herein; comprising the amino acid sequences of SEQ ID NOS: 2-7, SEQ ID NOS: 8 and 9 or SEQ ID NOS: 10 and 11), wherein the antibody is administered (a) at Q3w for one or more administrations and (b) at Q4w for one more administrations, wherein in each case the antibody is administered at a fixed dose of 3000 mg.

In one embodiment, provided is a method for administering an anti-CD39 antibody (e.g. an anti-CD39 antibody having the characteristics described herein; comprising the amino acid sequences of SEQ ID NOS: 2-7, SEQ ID NOS: 8 and 9 or SEQ ID NOS: 10 and 11), wherein the antibody is administered Q3w and/or Q4w at a fixed dose of 2250 mg. In one embodiment, the anti-CD39 antibody is administered Q3w at a fixed dose of 2250 mg as neoadjuvant therapy and Q4w at a dose of 2250 mg as adjuvant therapy. In another embodiment, provided is a method for administering an anti-CD39 antibody (e.g. an anti-CD39 antibody having the characteristics described herein; comprising the amino acid sequences of SEQ ID NOS: 2-7, SEQ ID NOS: 8 and 9 or SEQ ID NOS: 10 and 11), wherein the antibody is administered (a) at Q3w for one or more administrations and (b) at Q4w for one more administrations, wherein in each case the antibody is administered at a fixed dose of 2250 mg.

Optionally, in any embodiment herein, a method is characterized as a method of treating cancer and/or preventing recurrence of cancer. Optionally, the method is characterized as a method of administering an anti-CD39 antibody in combination with an anti-PD(L)1 antibody (and/or chemotherapy). Optionally, in any embodiment, the method is characterized as a method of administering an anti-CD39 antibody (and optionally further an anti-PD(L)1 antibody, and optionally further chemotherapy) in combination with surgery and optionally further radiation therapy.

Preferably, the anti-CD39 antibody is administered intravenously (i.v.). Preferably, the anti-PD(L)1 antibody is administered intravenously.

In some embodiments, the dose of anti-PD(L)1 antibody and the chemotherapy to be administered to the patient can vary depending, in part, upon the size (body weight, body surface, or organ size) and condition (the age and general health) of the patient.

In some embodiments, the anti-PD(L)1 antibody is durvalumab and is administered at a fixed dose of 1500 mg. Durvalumab is commercialized as Imfinzi™ by AstraZeneca.

A neoadjuvant therapy treatment regimen can thus comprise administering the anti-CD39 antibody at a fixed dose of 2250 mg or 3000 mg every 3 weeks and durvalumab at a fixed dose of 1500 mg every 3 weeks.

An adjuvant therapy treatment regimen can thus comprise administering the anti-CD39 antibody at a fixed dose of 2250 mg or 3000 mg every 4 weeks and administering durvalumab at a fixed dose of 1500 mg every 4 weeks.

In particular embodiments, the anti-CD39 antibody and the anti-PD(L)1 antibody, and optionally further the chemotherapy, are administered simultaneously, concurrently, separately, or sequentially. In some embodiments, anti-CD39 antibody and anti-PD(L)1 antibody are administered prior to the chemotherapy. In further embodiments, anti-CD39 antibody and anti-PD(L)1 antibody are administered concurrently with chemotherapy. Advantageously, the anti-CD39 antibody and the anti-PD(L)1 antibody are administered on the same day, e.g. the first day of the three week cycle or the first day of the four week cycle for neoadjuvant and adjuvant treatment, respectively. Optionally, for neoadjuvant treatment, the anti-CD39 antibody and the anti-PD(L)1 antibody are administered on the same day as an administration of chemotherapy (e.g. the first day of the three week cycle).

Accordingly, a neoadjuvant therapy can comprise one or more cycles of treatment with an administration of anti-CD39 antibody having the respective heavy and light chain amino acid sequence of SEQ ID NOS: 10 and 11 at a fixed dose of 2250 or 3000 mg and an administration of durvalumab at a fixed dose of 1500 mg, wherein the anti-CD39 antibody and durvalumab are administered once on the first day of a three week cycle. The neoadjuvant therapy can comprise for example 2, 3, 4 or more of such cycles of treatment.

An adjuvant therapy can comprise one or more cycles of treatment with an administration of anti-CD39 antibody having the respective heavy and light chain amino acid sequence of SEQ ID NOS: 10 and 11 at a fixed dose of 2250 or 3000 mg and an administration of durvalumab at a fixed dose of 1500 mg, wherein the anti-CD39 antibody and durvalumab are administered once on the first day of a four week cycle. The adjuvant therapy can comprise for example 2, 3, 4, 5, 6, 7, 8, 9, 10, 11, 12 or more of such cycles of treatment, or at least 2, 3, 4, 5, 6, 7, 8, 9, 10, 11, 12 cycles. In one embodiment, anti-CD39 antibody and durvalumab are administered as adjuvant therapy for up to 12 cycles.

Thus, when the anti-CD39 antibody and the anti-PD(L)1 antibody are used as neoadjuvant and adjuvant therapy, a treatment can thus comprise:
(a) administering as neoadjuvant therapy one or more administrations of anti-CD39 antibody having the respective heavy and light chain amino acid sequence of SEQ ID NOS: 10 and 11 at a fixed dose of 2250 or 3000 mg and durvalumab at a fixed dose of 1500 mg, wherein the anti-CD39 antibody and the durvalumab are administered on the first day of a three week cycle; and
(b) administering as adjuvant therapy one or more administrations of anti-CD39 antibody having the respective heavy and light chain amino acid sequence of SEQ ID NOS: 10 and 11 at a fixed dose of 2250 or 3000 mg and durvalumab at a fixed dose of 1500 mg, wherein the anti-CD39 antibody and durvalumab are administered on the first day of a four week cycle.

Adjuvant therapy anti-CD39 antibody and anti-PD(L)1 antibody can be initiated immediately following surgical resection of the tumor or cancer, and preferably the adjuvant therapy will be indicated within 10 weeks of surgical resection. The adjuvant therapy can continue for up to 12 cycles, or for more than 12 cycles, e.g. until disease progression or until a desired response is obtained (e.g. CR), or until ctDNA clearance.

In some embodiments, the anti-CD39 antibody and anti-PD(L)1 antibody are used (e.g. as neoadjuvant therapy and optionally adjuvant therapy) independently or irrespectively the mutation status of epidermal growth factor receptor (EGFR) and/or anaplastic lymphoma kinase (ALK) genes. In some embodiments, the anti-CD39 antibody and anti-PD(L)1 antibody are used to treat patients lacking EGFR mutations (EGFR wild type) and/or lacking ALK gene rearrangements (ALK wild type).

In some embodiments, the anti-CD39 antibody and anti-PD(L)1 antibody are used (e.g. as neoadjuvant therapy and optionally adjuvant therapy) independently or irrespectively of the PD-L1 expression status of the patient. In some embodiments, the anti-CD39 antibody and anti-PD(L)1 antibody are used as neoadjuvant therapy and optionally adjuvant therapy for the treatment of patients that has detectable PD-L1 expression, for example a patient having a CPS score of at least 1 (CPS≥1).

PD-L1 expression can include any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent.

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if it is at least 1% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the MS is ≥5.

Several PD-L1 protein scoring systems are commonly used. “Tumor proportion score (TPS)” refers to the percentage of tumor cells expressing PD-L1 on the cell membrane at any intensity (weak, moderate or strong). Linear partial or complete cell membrane staining is interpreted as positive for PD-L1. “Mononuclear inflammatory density score (MIDS)” refers to the ratio of the number of PD-L1 expressing mononuclear inflammatory cells (MIC) infiltrating or adjacent to the tumor (small and large lymphocytes, monocytes, and macrophages within the tumor nests and the adjacent supporting stroma) compared to the total number of tumor cells. The MIDS is recorded at a scale from 0 to 4 with 0=none; 1=present, but less than one MIC for every 100 tumor cells (<1%); 2=at least one MIC for every 100 tumor cells, but less than one MIC per 10 tumor cells (1-9%); 3=at least one MIC for every 10 tumor cells, but fewer MIC's than tumor cells (10-99%); 4=at least as many MIC's as tumor cells (≥100%). “Combined positive score (CPS)” refers to the ratio of the number of PD-L1 positive tumor cells and PD-L1 positive mononuclear inflammatory cells (MIC) within the tumor nests and the adjacent supporting stroma (numerator) compared to the total number of tumor cells (denominator; i.e., the number of PD-L1 positive and PD-L1 negative tumor cells). PD-L1 expression at any intensity is considered positive, i.e., weak (1+), moderate (2+), or strong (3+).

When the anti-CD39 antibody and anti-PD(L)1 antibody are used as adjuvant therapy (e.g. in addition to neoadjuvant therapy), the treatments of the disclosure may optionally be advantageous for a patient who is minimal residual disease-positive (MRD+) following surgical resection. For example, following neoadjuvant therapy and surgery, one can optionally (a) determining whether the patient is minimal residual disease-positive (MRD+); and (b) administering to the patient anti-CD39 antibody and anti-PD(L)1 antibody as adjuvant therapy if the patient is identified as MRD+. In one embodiment, the anti-CD39 antibody and anti-PD(L)1 antibody are administered within 10 weeks of tumor resection.

The MRD status of a patient can be determined using methods known in the art (see, e.g., Abbosh et al. (2017); Chaudhuri et al. (2017)). In some embodiments, a patient's MRD status can be determined using a multi-step assay. First, whole exome sequencing (WES) is performed on DNA extracted from the patient's tumor tissue and controlled for germline mutations by WES of the patient's whole blood. A personalized panel is then developed, comprised of the patient's tumor variants expressed at high frequency. This panel is then used to identify the presence of these variants on circulating tumor DNA (ctDNA) extracted from the patient's plasma and the patient is considered MRD+ if the panel detects a tumor variant. This personalized approach allows detection of the patient's tumor variants in DNA extracted from their plasma at high sensitivity.

In some embodiments, determining whether the patient is minimal residual disease positive (MRD+) is determined by: (a) sequencing all or part of the genome or exome of a tumor of the patient to define clonal and/or subclonal mutations in the tumor; (b) defining a set of reagents that will detect the presence of DNA from the tumor via the presence of the clonal and/or subclonal mutations; and (c) analyzing a sample comprising DNA from the tumor obtained from the patient subsequent to the tumor removal and the defined set of reagents to determine whether the tumor has recurred by detection of the clonal and/or subclonal mutations in the sample. The presence and/or rise of clonal and/or subclonal mutations in the sample from the patient characteristic of the tumor indicates whether the tumor has recurred. The clonal and/or subclonal mutations characteristic of the patient's tumor are defined by sequencing all or part of the whole genome and/or exome of DNA from the tumor, in certain instances after the tumor has been resected from the patient. Using a set of reagents designed or defined to detect the presence of DNA from the tumor via the presence of the specific clonal and/or subclonal mutations identified for the specific subject of interest, the presence and/or rise of clonal and/or subclonal mutations in the sample obtained from the patient is analyzed.

In some embodiments, the sequencing is carried out on a tumor biopsy, all or part of the tumor or one or more subsections of the tumor, cell free DNA (cfDNA), ctDNA, exosome derived tumor DNA, or circulating tumor cells from the subject. In some embodiments, the sequencing is carried out on the tumor or subsection thereof following removal of the tumor. In some embodiments, all or part of the genome or exome of at least two subsections of the tumor is sequenced and clonal and/or subclonal mutations are defined based on which mutations occur in which tumor subsections. In some embodiments, the defined set of reagents comprise multiplex PCR primers and the analysis is a multiplex PCR. In some embodiments, sequencing is carried out on blood plasma obtained from the patient, or the sample to be analyzed is a blood plasma sample from the patient.

MRD, as indicated by detection of ctDNA, may reveal the existence of clinically indiscernible residual tumor following curative intent therapy (surgery±chemotherapy/radiotherapy). Detection of MRD at a time when there is no radiologic evidence of disease provides an opportunity for earlier therapeutic intervention. Anti-CD39 antibody and anti-PD(L)1 antibody can for example be administered as soon as possible, and preferably within 10 weeks of tumor resection. MRD+ patients experience inferior recurrence-free survival compared to MRD-patients. Therefore, MRD+ patients may benefit from earlier intervention and escalation of treatment, including immunotherapy alone or in combination with chemotherapy; furthermore, MRD-patients (the majority of whom are cured by surgery alone) could be spared from more intensive therapy and the resulting unnecessary toxicity.

It will be appreciated that the anti-CD39 antibody and the anti-PD(L)1 antibody can be incorporated in a pharmaceutical formulation in a suitable concentration (e.g. from 1 mg/ml to 500 mg/ml, wherein said formulation has a pH from 2.0 to 10.0). The anti-CD39 antibody and the anti-PD(L)1 antibody can be comprised in the same or separate pharmaceutical formulations. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

Optionally, the pharmaceutical formulation is a freeze-dried formulation, whereto the physician or the patient adds solvents and/or diluents prior to use. Optionally, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0. Optionally, the pH of the formulation is in the range selected from the list consisting of from about 2.0 to about 10.0, about 3.0 to about 9.0, about 4.0 to about 8.5, about 5.0 to about 8.0, and about 5.5 to about 7.5. In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19^{th} edition, 1995.

It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Administration of pharmaceutical compositions according to the invention may be through any of several routes of administration, for example, intravenous. Suitable antibody formulations can also be determined by examining experiences with other already developed therapeutic monoclonal antibodies.

In one aspect, provided herein are kits, for example kits which include:
(i) a pharmaceutical composition containing an anti-CD39 antibody such as an anti-CD39 antibody comprising the VH and VL amino acid sequences of SEQ ID NOS: 8 and 9 respectively, and an anti-PD(L)1 antibody such as durvalumab, or
(ii) a first pharmaceutical composition containing an anti-PD(L)1 antibody such as durvalumab, and a second pharmaceutical composition containing an anti-CD39 antibody such as an anti-CD39 antibody comprising the VH and VL amino acid sequences of SEQ ID NOS: 8 and 9, respectively, or
(iii) a pharmaceutical composition containing an anti-CD39 antibody such as an anti-CD39 antibody comprising the respective VH and VL amino acid sequences of SEQ ID NOS: 8 and 9, and instructions to administer said anti-CD39 antibody with an anti-PD(L)1 antibody (e.g. durvalumab), for example as neoadjuvant and/or adjuvant therapy, optionally for NSCLC, or
(iv) a pharmaceutical composition containing an anti-PD(L)1 antibody (e.g. durvalumab), and instructions to administer said anti-PD(L)1 antibody with an anti-CD39 antibody (e.g., anti-CD39 antibody comprising the respective VH and VL amino acid sequences of SEQ ID NOS: 8 and 9), for example as neoadjuvant and/or adjuvant therapy, optionally for NSCLC.

In any embodiment, a kit can optionally further comprise a chemotherapy, e.g. a chemotherapy comprising a platinum agent, gemcitabine, including combinations of chemotherapy agents, for example carboplatin and paclitaxel, cisplatin and gemcitabine, cisplatin and pemetrexed, or carboplatin and pemetrexed.

A pharmaceutical composition may optionally be specified as comprising a pharmaceutically-acceptable carrier. An anti-CD39 antibody or an anti-PD(L)1 antibody may optionally be specified as being present in a therapeutically effective amount adapted for use in any of the methods herein. An anti-CD39 antibody may optionally be specified as comprising the CDR amino acid sequences of SEQ ID NOS: 2-7. An anti-CD39 antibody may optionally be specified as comprising the heavy and light chain amino acid sequences of SEQ ID NOS: 10 and 11. The kits optionally also can include instructions, e.g., comprising administration schedules (e.g. the Q3w and/or Q4w schedules disclosed herein), to allow a practitioner (e.g., a physician, nurse, or patient) to administer the composition contained therein to a patient having cancer (e.g., a solid tumor, in particular an NSCLC, a Stage II or III NSCLC, an NSCLC that is resectable or has not yet been surgically resected). In any embodiment, a kit optionally can include instructions to administer said anti-CD39 antibody simultaneously, separately, or sequentially with said anti-PD(L)1 antibody. The instructions can optionally further specify administration of said anti-CD39 antibody and said anti-PD(L)1 antibody simultaneously, separately, or sequentially with chemotherapy (e.g. for neoadjuvant therapy). The kit also can include a syringe.

A kit can be specified as comprising one or more containers (e.g. single use vials or pre-filled syringes) comprising the specified pharmaceutical composition or antibody.

A specified amount or dose (e.g. 3000 mg, 2250 or 1500 mg) can be specified as being provided in a plurality of vials. For example, a vial of anti-CD39 antibody can comprise 375 mg of anti-CD39 antibody. A vial of durvalumab can contain 120 mg or 500 mg of durvalumab (e.g. 120 mg in 2.4 mL or 500 mg in 10 mL).

Optionally, the kits include multiple packages of the single-dose pharmaceutical compositions each containing an effective amount of the anti-CD39 antibody, and/or the anti-PD(L)1 antibody, for a single administration in accordance with the methods provided above. Instruments or devices necessary for administering the pharmaceutical composition(s) also may be included in the kits. For instance, a kit may provide one or more pre-filled syringes containing an amount of the anti-CD39 antibody or anti-PD(L)1 antibody.

In one embodiment, the present invention provides a kit for treating a cancer or a tumor in a human patient, the kit comprising one or more single-use vials comprising an anti-CD39 antibody comprising the H-CDR1, H-CDR2 and H-CDR3 domains of a heavy chain variable region having the sequence set forth in SEQ ID NO: 8, and the L-CDR1, L-CDR2 and L-CDR3 domains of a light chain variable region having the sequence set forth in SEQ ID NO: 9, and optionally, instructions for using said anti-CD39 antibody in any of the methods described herein (e.g. at a dosage, frequency described herein).

In one embodiment, the present invention provides a kit for treating a cancer or a tumor in a human patient, optionally wherein said cancer or tumor is an NSCLC (e.g. a stage II or III NSCLC, a resectable NSCLC, etc.), the kit comprising:
(a) one or more vials comprising an anti-CD39 antibody comprising the H-CDR1, H-CDR2 and H-CDR3 domains of a heavy chain variable region having the sequence set forth in SEQ ID NO: 8, and the L-CDR1, L-CDR2 and L-CDR3 domains of a light chain variable region having the sequence set forth in SEQ ID NO: 9; and/or
(b) one or more vials comprising an anti-PD-L1 antibody, optionally a dose of durvalumab; and
(c) optionally, instructions for using said anti-CD39 antibody and/or said anti-PD(L)1 antibody in any of the methods described herein.

In one embodiment, a vial of anti-CD39 antibody contains 375 mg of anti-CD39 antibody. In one embodiment a kit comprises one or multiple sets of 6 vials of anti-CD39 antibody. In one embodiment a kit comprises one or multiple sets of 8 vials of anti-CD39 antibody. In one embodiment a kit comprises 4 sets of 6 or 8 vials of anti-CD39 antibody for use in neoadjuvant therapy. In one embodiment a kit comprises at least 4 sets (e.g. 4, 6, 8, 10 or 12 sets) of 6 or 8 vials of anti-CD39 antibody for use in adjuvant therapy.

In one embodiment, a vial of durvalumab contains 500 mg of durvalumab. In one embodiment a kit comprises one or multiple sets of 3 vials of durvalumab. In one embodiment a kit comprises 4 sets of 3 vials of durvalumab for use in neoadjuvant therapy. In one embodiment a kit comprises at least 4 sets (e.g. 4, 6, 8, 10 or 12 sets) of 3 vials of durvalumab for use in adjuvant therapy.

In one embodiment, the present invention provides a kit for treating a cancer or a tumor in a human patient, optionally wherein said cancer or tumor is an NSCLC (e.g. a stage II or III NSCLC, a resectable NSCLC), the kit comprising:
(a) a dose of an anti-CD39 antibody comprising the H-CDR1, H-CDR2 and H-CDR3 domains of a heavy chain variable region having the sequence set forth in SEQ ID NO: 8, and the L-CDR1, L-CDR2 and L-CDR3 domains of a light chain variable region having the sequence set forth in SEQ ID NO: 9; and/or
(b) a dose of an anti-PD-L1 antibody, optionally a dose of durvalumab; and
(c) optionally, instructions for using said anti-CD39 antibody and/or said anti-PD(L)1 antibody in any of the methods described herein.

In one embodiment, the dose of anti-CD39 antibody can be a fixed dose of 2250 mg or 3000 mg. In one embodiment, the dose of durvalumab can be a fixed dose of 1500 mg.

In one embodiment, the present invention provides a kit, e.g. for treating a cancer or a tumor in a human patient, optionally wherein said cancer or tumor is an NSCLC (e.g. a stage II or III NSCLC, a resectable NSCLC), the kit comprising:
(a) one or more containers (e.g. vial(s)) together comprising 3000 mg or 2250 mg of an anti-CD39 antibody comprising the H-CDR1, H-CDR2 and H-CDR3 domains of a heavy chain variable region having the sequence set forth in SEQ ID NO: 8, and the L-CDR1, L-CDR2 and L-CDR3 domains of a light chain variable region having the sequence set forth in SEQ ID NO: 9; and/or
(b) one or more containers (e.g. vial(s)) together comprising 1500 mg of durvalumab; and
(c) optionally, instructions for using said anti-CD39 antibody and/or said anti-PD(L)1 antibody in any of the methods described herein.

In any embodiment, the instructions can specify that the anti-CD39 antibody is administered Q3w or Q4w. In one embodiment, the instructions specify that the anti-CD39 antibody is administered Q3w as neoadjuvant therapy and Q4w as adjuvant therapy. In one embodiment, the instructions specify that the anti-PD(L)1 antibody is administered as Q3 or Q4. In one embodiment, the instructions specify that the anti-PD(L)1 antibody is administered Q3w as neoadjuvant therapy and Q4w as adjuvant therapy. In one embodiment, the instructions specify that the anti-CD39 antibody and the anti-PD(L)1 antibody are administered on the same days (e.g. on day 1 of a 3 week cycle in the neoadjuvant setting and on day 1 of a 4 week cycle in the adjuvant setting). In one embodiment, the instructions specify that the anti-CD39 antibody and the anti-PD(L)1 antibody are administered for 4 cycles as neoadjuvant therapy. In one embodiment, the instructions specify that the anti-CD39 antibody and the anti-PD(L)1 antibody can be administered in combination with chemotherapy in the neoadjuvant setting. An anti-CD39 antibody may optionally be specified as comprising heavy and light chain variable region amino acid sequences of SEQ ID NOS: 8 and 9, or the heavy and light chain amino acid sequences of SEQ ID NOS: 10 and 11.

Optionally, the kit further comprises a dose of a chemotherapy, e.g. a chemotherapy comprising a platinum agent, gemcitabine, including combinations of chemotherapy agents, for example carboplatin and paclitaxel, cisplatin and gemcitabine, cisplatin and pemetrexed, or carboplatin and pemetrexed.

### EXAMPLES

### Example 1: CD39 Expression in Early and Late-Stage NSCLC Biopsies

CD39 staining was performed with antibody EPR20627 clone from Abcam on 50 squamous cell NSCLC (sqNSCLC) and 50 adenocarcinoma NSCLC (adNSCLC) FFPE samples. CD39 expression scoring (0-12) is a combination of staining frequency (0-4) and intensity (1-3).

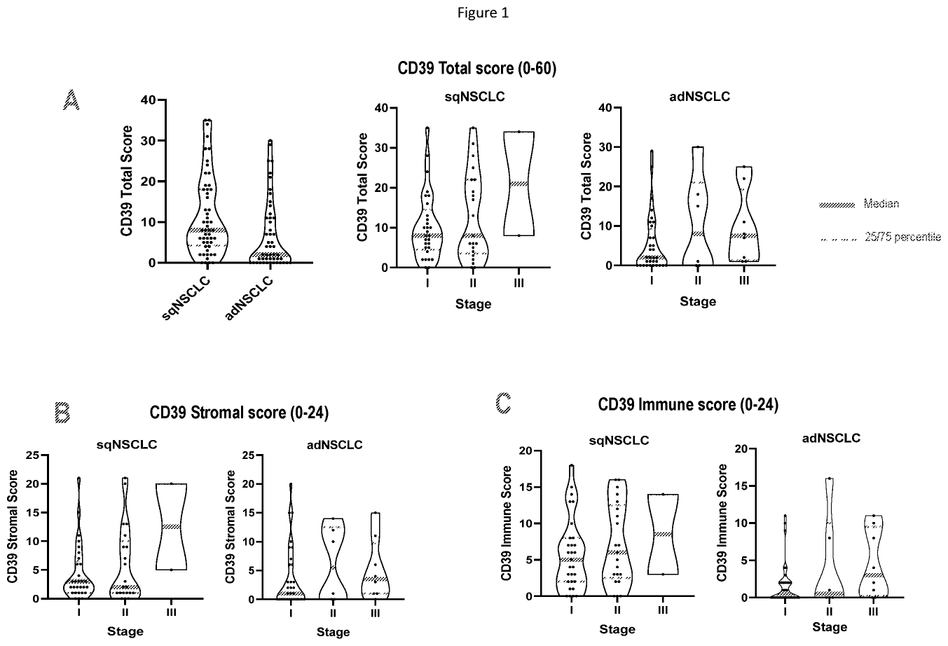

Results are shown in FIG. 1: Panel A shows CD39 total score is the sum of the stromal, immune and tumor expression scores (0-60). Panel B shows Stromal score is the sum of the vascular (0-12) and connective tissue (0-12) expression scores. Panel C shows Immune score as the sum of the small immune cell (0-12) and large immune cell (0-12) scores. In each case, expression scores are shown by cancer stages (Stage I, II or III).

Both sqNSCLC and adNSCLC shows staining on stromal and immune cells, including in early stage biopsies. Total score was higher in sqNSCLC. No or poor CD39 staining was observed on tumor cells.

### Example 2: ATP Release from Squamous NSCLC Tumor Cells Following Chemotherapy Treatment

This experiment aimed as studying ATP release from squamous NSCLC tumor cells following chemotherapy treatment. The squamous NSCLC H1703 tumor cell line was incubated separately with different chemotherapies. The measurement of ATP release was performed 24 h after Cisplatin treatment, 72 h after Carboplatin, 60 h after Oxaliplatin, 48 h after Pemetrexed, 48 h after Gemcitabine, 72 h after 5-FU, 24 h after Paclitaxel and 40 h after Docetaxel. Extracellular ATP release in the culture supernatant was measured with a luminescent-based assay (CellTiter-Glo®) and expressed in Luminescence Arbitrary Unit (AU). Means+/−SD.

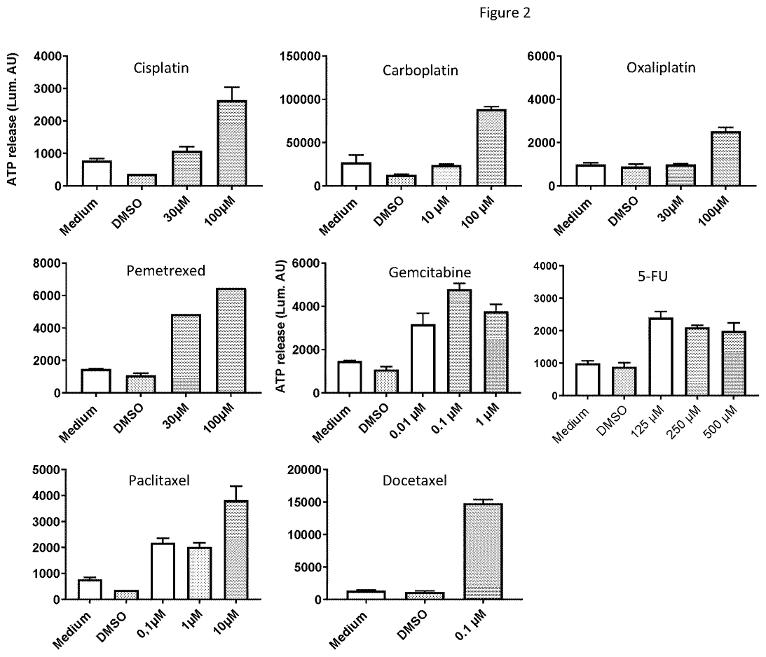

Result are shown in FIG. 2, showing ATP release from H1703 tumor cell line (sqNSCLC) following the treatment with different chemotherapies, cisplatin, carboplatin, oxaliplatin, pemetrexed, gemcitabine, 5-Fu, paclitaxel and docetaxel. Each of the chemotherapy agents induced significant ATP release in the NSCLC tumor cell line.

### Example 3: IPH5201 was Able to Accumulate the Released ATP, Following Chemotherapy Treatment

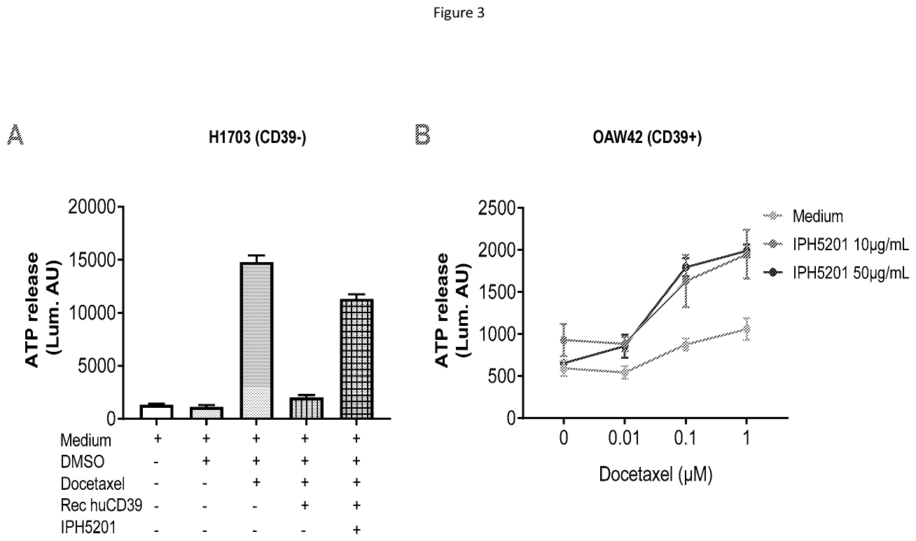

H1703 (CD39−) cells were incubated with recombinant human CD39 protein (huCD39) (400 ng/ml) to mimic soluble CD39 in tumor microenvironment with or without IPH5201 (humanized anti-CD39 antibody having the respective heavy and light chain amino acid sequences of SEQ ID NOS: 10 and 11) at 10 μg/mL and then treated with docetaxel (0.1 μM). The measure of extracellular ATP (eATP) release was performed 40 h after docetaxel treatment. Results are shown in FIG. 3A. In the H1703 cells that do not express CD39, docetaxel induced strong release of eATP, which was reduced in the presence of added recombinant human CD39 protein, and then restored in the presence of added IPH5201 antibody.

OAW42 (CD39+) cells were incubated with 10 or 50 μg/mL of IPH5201 and treated with a dose range of docetaxel. The measure of eATP release was performed 30 h after docetaxel treatment. eATP was measured in the cell culture supernatant with a luminescent-based assay (CellTiter-Glo®) and expressed in Luminescence Arbitrary Unit (AU). Means+/−SD. Results are shown in FIG. 3B. In the OAW42 cells that express CD39, docetaxel induced at best a modest release of eATP at the highest concentrations, however in the presence of 10 or 50 μg/mL of IPH5201, eATP accumulated significantly.

### Example 4: CD39 is Expressed in the Tumor Microenvironment in Mouse Models and molPH5201 Significantly Reduces Adenosine Levels in Tumors In Vivo

Expression of human CD39 in MCA205 tumors engrafted in human CD39 knock-in (huCD39KI) mice was assessed, followed by measure of intratumoral levels of adenosine after in vivo treatment with molPH5201, an antibody having the VH and VL or SEQ ID NO NOS: 8 and 9 and murine constant domains including an Fc domain with amino acid substitutions to eliminate binding to mouse Fcγ receptors, in order to evaluate the reduction of adenosine levels in tumors by molPH5201 can reduce in vivo.

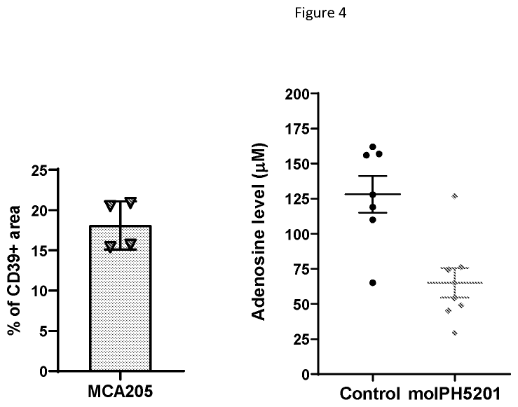

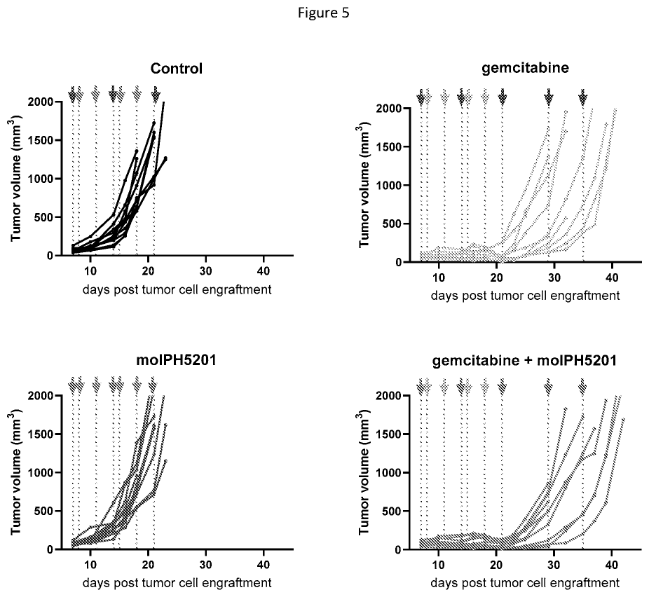

Human CD39 expression on MCA205 tumors harvested on day 16 post engraftment was evaluated by IHC with EPR20627 clone from Abcam B&C. MCA205 tumors were engrafted in huCD39KI mice. On day 7 and 14 post tumor implantation, they were treated with molPH5201 (20 mg/kg) or isotype control. On day 16, tumors were harvested. Results are shown in FIG. 4, left hand panel, showing that a significant percentage of cells express CD39.

Quantification of adenosine in the harvested MCA205 tumors was performed using pre-column derivatization and liquid chromatography-mass spectrometry (LC-MS/MS) as described by Goodwin et al. 2019 Anal Biochem 568:78-88. Results are shown in FIG. 4, right hand panel, showing that compared to control, molPH5201-treated animals showed less intratumoral adenosine.

Blockade of CD39 enzymatic activity was determined using the Wachstein-Meisel CD39 enzyme assay (brown color indicates enzyme activity). Tissues from molPH5201-treated animals showed less brown color indication decreased CD39 enzymatic activity.

### Example 5: Anti-CD39 Improves the Anti-Tumor Efficacy of Chemotherapy and Anti-PD-L1 In Vivo

In vivo experiments were performed in human CD39 knock-in (huCD39KI) mice, genetically modified to express human CD39 in place of its murine counterpart mouse CD39. These mice were sub-cutaneously (s.c.) engrafted with the MC38 cancer cell line. Mice bearing established tumors were dosed with isotype control (IC) antibodies, anti-human CD39 antibody (molPH5201, described above), gemcitabine chemotherapy and anti-mouse PD-L1 antibody (an antibody that binds murine PD-L1 having an Fc domain with amino acid substitutions to eliminate binding to mouse Fcγ receptors.

In Vivo Anti-Tumor Efficacy of molPH5201 and Gemcitabine

Male and female huCD39KI mice were engrafted subcutaneously (s.c.) with 1×10⁶ MC38 cancer cells. Treatments were initiated after mouse randomization. Mice were treated twice a week for two weeks with gemcitabine (25 mg/kg) or PBS (at day 8, 11, 15 and 18 after cell engraftment) and once a week for four weeks with either anti-human CD39 antibody (molPH5201) or the corresponding isotype control (IC) antibody (400 μg/mouse).

Randomization of mice into four groups was performed on day 7 post tumor cell engraftment, at a mean tumor volume of 72 mm³±27 mm³ with individual tumor volumes comprised between 37 and 130 mm³.

Results are shown in FIG. 5. Both gemcitabine alone and the combination of gemcitabine and molPH5201 drastically and significantly slowed down tumor growth compared to the control group (p<0.001). molPH5201 did not modify tumor growth as compared to the control and did not improve the anti-tumor efficacy of gemcitabine. No complete response was observed in this experiment. No evidence of over toxicity was observed in any group.

In Vivo Anti-Tumor Efficacy of Chemotherapy, molPH5201 and Anti-PD-L1 Antibody

The anti-tumor effects of different combinations of treatment with gemcitabine, molPH5201 and anti-PD-L1 antibody were evaluated as a combined treatment. Three independent experiments were performed to assess this triple combination.

### Experiment 1

Male and female huCD39KI mice were engrafted s.c. with 1×10⁶ MC38. Treatments were initiated after mouse randomization. Mice were treated twice a week for two weeks with gemcitabine (25 mg/kg) or PBS, twice a week for three weeks with either anti-mouse PD-L1 antibody or the corresponding IC antibody (200 μg/mouse) and once a week for four or five weeks depending on the experiment with either anti-human CD39 antibody (molPH5201) or the corresponding IC antibody (400 μg/mouse). Mouse randomization into five groups was performed on day 7 post tumor cell engraftment at a mean tumor volume of 62 mm³±23 mm³ with individual tumor volumes comprised between 31 and 117 mm³.

No evidence of increased toxicity was observed in any group. One mouse in the gemcitabine+molPH5201 group lost more than 10% of body weight but recovered its initial weight progressively after the last gemcitabine treatment.

Gemcitabine either as single agent or in all three tested combinations significantly slowed down tumor growth compared to the control (p<0.001). Neither anti-PD-L1 nor molPH5201 improved the anti-tumor effect of gemcitabine as single agents, while the addition of both anti-PD-L1+molPH5201 to gemcitabine induced a significantly better efficacy than gemcitabine alone (p<0.05 when control group was excluded). Among the groups, molPH5201 did not statistically significantly improve gemcitabine+anti-PD-L1 efficacy and anti-PD-L1 did not statistically significantly improve the anti-tumor effect of gemcitabine+molPH5201.

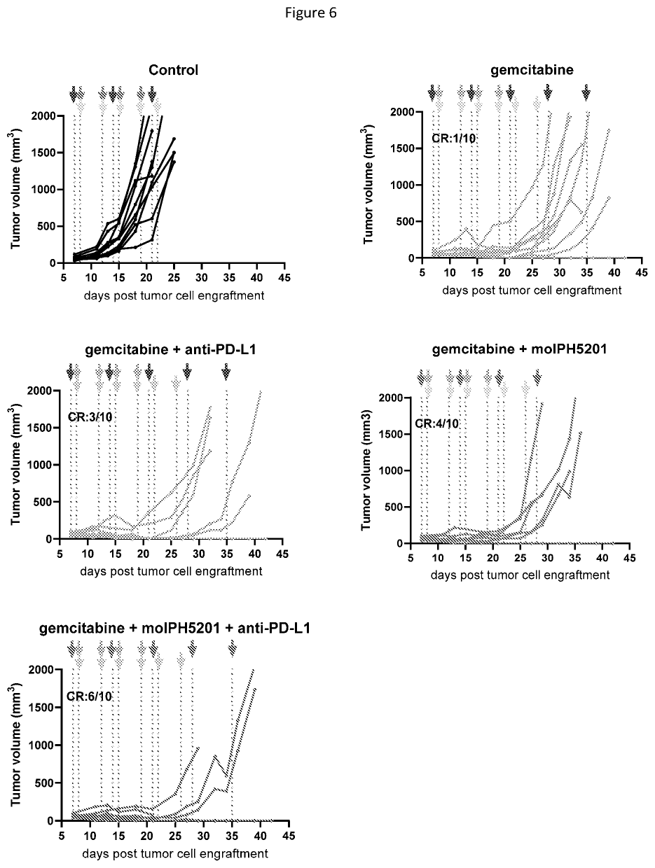

Results are shown in FIG. 6. Both anti-PD-L1 and molPH5201 agents increased the number of complete response when combined with gemcitabine in comparison to gemcitabine alone. Moreover a benefit of molPH5201 on the anti-tumor effect of gemcitabine+anti-PD-L1 was supported by the proportion of complete responses (CRs): 6/10 (60%) were observed in the group gemcitabine+anti-PD-L1+molPH5201 and 3/10 (30%) in the group treated with gemcitabine+anti-PD-L1. Arrows indicate administration of the different agents.

### Experiment 2

As in Experiment 1, male and female huCD39KI mice were engrafted s.c. with 1×10⁶ MC38. Treatments were initiated after mouse randomization. Mice were treated twice a week for two weeks with gemcitabine (25 mg/kg) or PBS, twice a week for three weeks with either anti-mouse PD-L1 antibody or the corresponding IC antibody (200 μg/mouse) and once a week for four or five weeks depending on the experiment with either anti-human CD39 antibody (molPH5201) or the corresponding IC antibody (400 μg/mouse). Mouse randomization into five groups was performed on day 7 post tumor cell engraftment at a mean tumor volume of 64 mm³±23 mm³ with individual tumor volumes comprised between 31 and 115 mm³.

As in the previous experiment, gemcitabine as single agent and in combination therapy significantly slowed down tumor growth compared to the control (p<0.001). No evidence of over toxicity was observed in any group.

Interestingly, when the untreated control group was excluded from the statistical analysis, molPH5201 tended to improve the anti-tumor efficacy of the combination gemcitabine+anti-PD-L1 (difference marginally significant, p<0.1). Similarly to previous experiment, the addition of both anti-PD-L1⁺molPH5201 to gemcitabine induced a better efficacy than gemcitabine alone (difference marginally significant p<0.1, when control group was excluded).

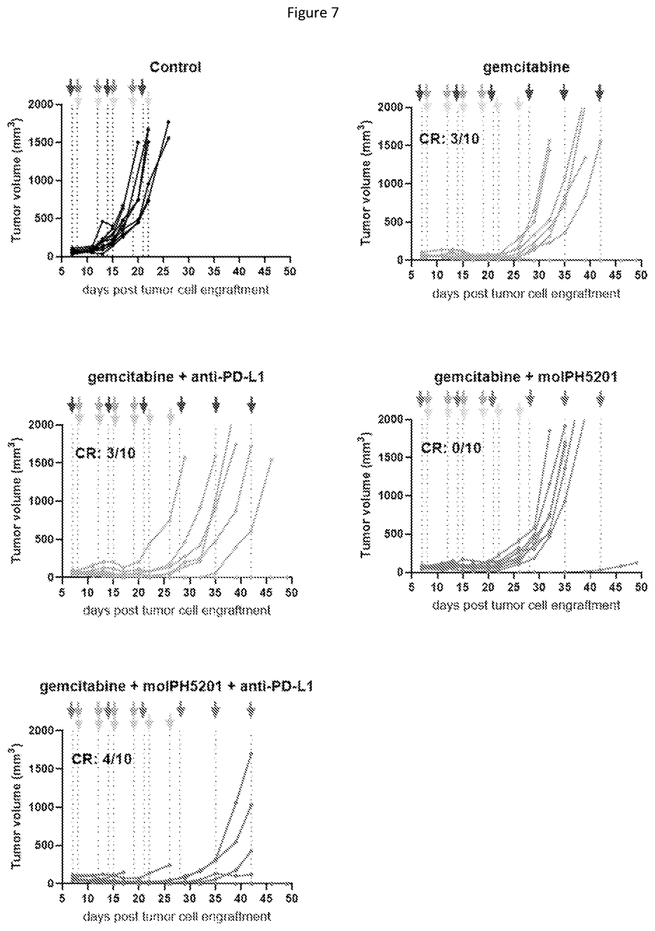

Results are shown in FIG. 7. The proportions of CRs were 4/10 in the group treated with the triple combination versus 3/10 in the group treated with gemcitabine+anti-PD-L1. In view of these results, this experiment was reproduced a third time with the same setting and results are shown in the next section.

### Experiment 3

As in Experiments 1 and 2, male and female huCD39KI mice were engrafted s.c. with 1×10⁶ MC38. Treatments were initiated after mouse randomization. Mice were treated twice a week for two weeks with gemcitabine (25 mg/kg) or PBS, twice a week for three weeks with either anti-mouse PD-L1 antibody or the corresponding IC antibody (200 μg/mouse) and once a week for four or five weeks depending on the experiment with either anti-human CD39 antibody (molPH5201) or the corresponding IC antibody (400 μg/mouse). Mouse randomization into five groups was performed on day 7 post tumor cell engraftment, at a mean tumor volume of 74 mm³±21 mm³ with individual tumor volumes comprised between 40 and 121 mm³. No sign of over toxicity was observed in any of the groups.

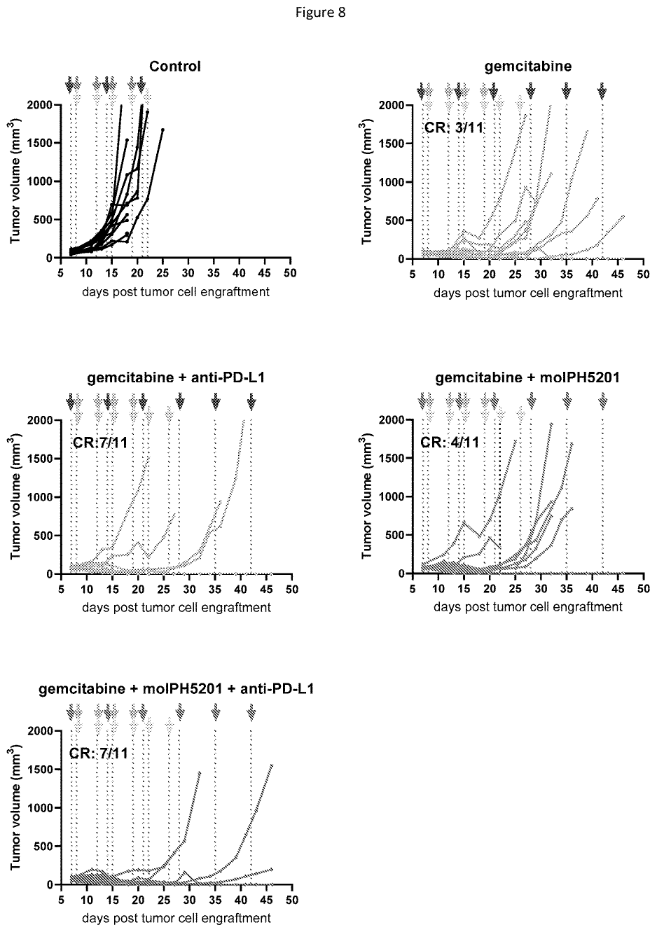

Results are shown in FIG. 8. As in both previous experiments, gemcitabine as single agent and in all combination tested significantly inhibited tumor growth compared to the control (p<0.001). Neither anti-PD-L1 nor molPH5201 improved the anti-tumor effect of gemcitabine as single agents, however the addition of both anti-PD-L1+molPH5201 to gemcitabine induced a better efficacy over gemcitabine alone (difference marginally significant p<0.1, when control group was excluded). molPH5201 did not statistically significantly improve gemcitabine+anti-PD-L1 efficacy. Thus, in this experiment, anti-PD-L1 increased the number of complete response when combined with gemcitabine, in comparison to gemcitabine alone, but molPH5201 did not increase the number of CR when combined to gemcitabine alone or to gemcitabine+anti-PD-L1.

### Pooled Analysis

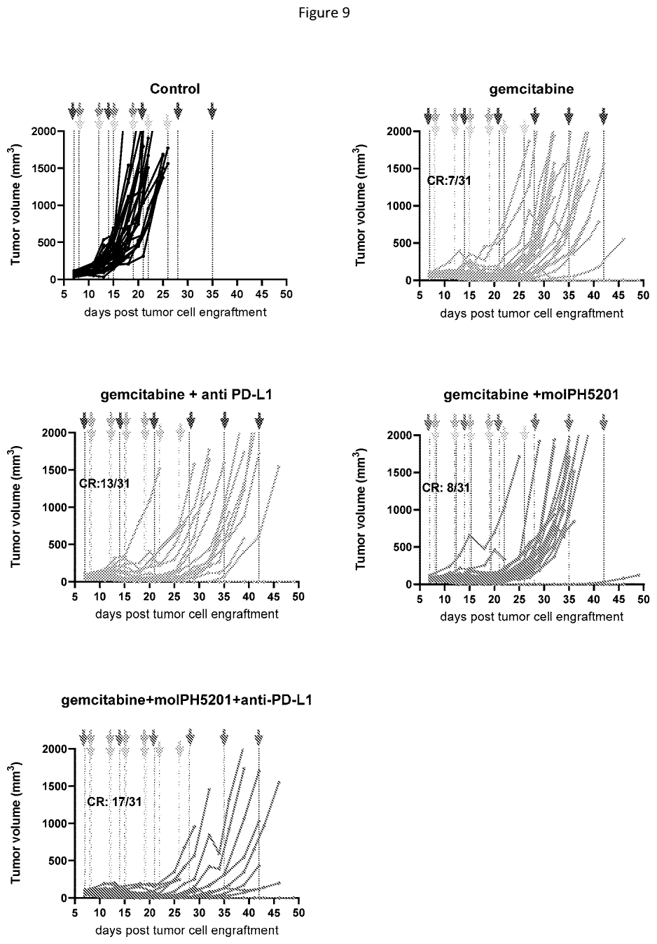

The three experiments were pooled and results are shown in FIG. 9, showing Tumor growth of MC38 tumor in huCD39KI mice post treatment with gemcitabine+/−anti-PD-L1+/−molPH5201. MC38 tumor-bearing huCD39KI mice were randomized on day 7 and then treated as indicated with 25 mg/kg ip of gemcitabine or PBS, 200 μg ip of anti-mouse PD-L1 antibody or corresponding isotype control antibody and with 400 μg iv of molPH5201 or corresponding IC antibody. Graphs show tumor growth in each individual (n=31/group). CR: Complete Response.

The pooled experiments performed in the MC38 tumor model (n=3), responsive to gemcitabine chemotherapy, showed that, while neither anti-PD-L1 nor molPH5201 improved the anti-tumor effect of gemcitabine as single agents (no statistically significant difference), the anti-PD-L1+molPH5201+gemcitabine triple combination improved the anti-tumor efficacy when compared to gemcitabine alone (p<0.05). Moreover, molPH5201 improved the anti-tumor efficacy of the gemcitabine+anti-PD-L1 combination (p<0.05. linear-mixed effect model analysis). This therapeutic benefit was also supported by the proportions of complete responses: 55% (17/31) were observed in the group of mice treated with the triple combination whereas only 42% (13/31) were observed in the group of mice treated with gemcitabine+anti-PD-L1.

In summary, IPH5201 blocks CD39 enzymatic activity, lowers adenosine intratumoral levels and increases extracellular ATP release by tumor cells upon chemotherapy treatment, and ultimately improves anti-tumor efficacy in preclinical models in combination with chemotherapy and blocking anti-PDL1 antibody. Altogether, the expression profile of CD39 in early-stage NSCLC and preclinical combination data support the clinical evaluation of IPH5201 in combination with durvalumab and chemotherapies in early-stage NSCLC patients.

### Example 6: Results from a Phase 1 Human Trial of Anti-CD39 Antibody and Design of a New Treatment Regimen for Anti-CD39 for Human Therapy

A first-in-human, multicentre, non-randomised, open-label, phase 1 study was conducted in order to assess the safety, efficacy, pharmacokinetics (PK) and pharmacodynamics (PD) of IPH5201±durvalumab (Imfinzi™) in patients with advanced solid tumours. The study consisted of two consecutive dose-escalation parts:
Part 1: Ascending doses of IPH5201 (100, 300, 1000 and 3000 mg) every 3 weeks (Q3W).
Part 2: Ascending doses of IPH5201 (300, 1000 and 3000 mg) and 1500 mg durvalumab Q3W.

PD cohorts enrolled patients with advanced squamous cell lung carcinoma or advanced pancreatic ductal adenocarcinoma in the top two dose levels of Parts 1 and 2. Primary endpoints were safety and tolerability. Key secondary endpoints included preliminary antitumour activity measured by objective response and disease control per RECIST v1.1, PK and immunogenicity. Exploratory endpoints included efficacy measured by duration of response and progression-free survival (PFS) per RECIST v1.1, overall survival and assessment of biomarkers.

Key inclusion criteria were:
Adults aged ≥18 years.
Histologically or cytologically confirmed advanced solid tumours.
At least one measurable lesion per RECIST v1.1.
Refractoriness to standard therapy or disease for which no standard therapy exists.
Eastern Cooperative Oncology Group Performance Status of 0 or 1.
Archival or fresh tumour sample.
Key exclusion criteria were:
Previous treatment with any agents targeting CD73, CD39, or adenosine receptors.
Treatment with any conventional or investigational anticancer therapy within 21 days of the first planned dose.
Active or prior autoimmune or inflammatory disorders within the past 5 years.
Cardiac and vascular criteria including presence of acute coronary syndrome or thromboembolic events within 6 months prior to enrolment, congestive heart failure, serious cardiac arrhythmia requiring medication, or uncontrolled hypertension.
Untreated metastases to the central nervous system.

### PK and PD Modelling

PK/PD modelling and simulation were used to guide selection of doses. IPH5201 PK data from 45 patients and PD data of free membrane CD39 (mCD39) occupancy on monocytes from 43 patients were available. A population PK model was developed with IPH5201 PK best described as a 2-compartment model with parallel linear and saturatable kinetics.

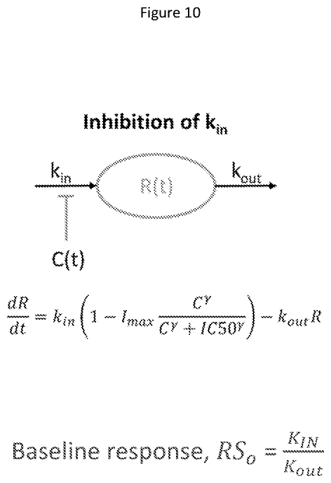

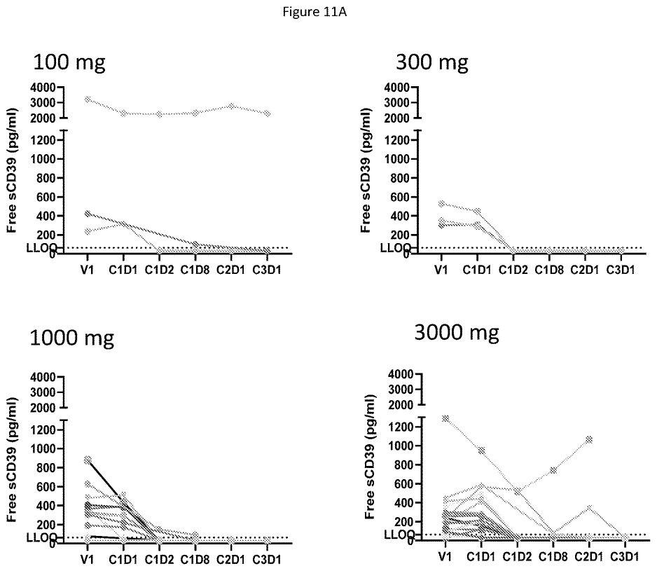

An indirect response PD model (inhibition on Kᵢₙ) was then built to describe the relationship between IPH5201 concentrations and free mCD39 on monocytes (FIG. 10). Increasing drug IPH5201 concentration decreases CD39free+ on monocytes (%) and the model parameters were listed in the table below. The model accurately predicted CD39free+ of monocytes (%). Maximum inhibition of CD39free+, Imax=0.926; IC50=2.44 μg/mL defines that IPH5201 produces 50% of maximum inhibition. The final indirect response model was then used for parametric simulation.

| Parameters | value | % RSE |
|---|---|---|
| Production rate constant (1/h) | 20.5 | 9% |
| IC50 (ug/mL) | 2.44 | 8% |
| Maximum inhibition (IMAX) | 0.92 | 1% |
| Baseline response (%) | 92 | FIX |
| Proportional error | 0.57 | 0.3% |
| IIV on KIN | 70.6% | 9% |
| IIV on IMAX | 5.9% | 5% |
| IIV on RS0 | 15.2% | 34% |

A total of 499 simulations using the model were conducted to predict mCD39 occupancy on monocytes and the proposed dose of Q3W or Q4W that would achieve at least 85% occupancy in the majority of patients and maintain sufficient exposure time at steady state was selected.

| | | 100% | | 90% | | 85% | |
|---|---|---|---|---|---|---|---|
| | | of the duration | | of the duration | | of the duration | |
| Total N = 499 | | | % | | % | | % |
| SCENARIO | | N | Subject | N | Subject | N | Subject |
| Q3W | 750 mg | 290 | 58% | 321 | 64.2% | 345 | 69% |
| | 1000 mg | 384 | 76.8% | 403 | 80.6% | 411 | 82.2% |
| | 1500 mg | 445 | 89% | 455 | 91% | 458 | 91.6% |
| | 2000 mg | 464 | 92.8% | 468 | 93.6% | 470 | 94% |
| | 3000 mg | 475 | 95% | 476 | 95.2% | 477 | 95.4% |
| Q4W | 750 mg | 91 | 18.2% | 133 | 26.6% | 156 | 31.2% |
| | 1000 mg | 207 | 41.4% | 257 | 51.4% | 290 | 58% |
| | 1500 mg | 362 | 72.4% | 393 | 78.6% | 408 | 81.6% |
| | 2000 mg | 420 | 84% | 436 | 87.2% | 445 | 89% |
| | 3000 mg | 459 | 91.8% | 465 | 93% | 468 | 93.6% |

At 3000 mg q3w, 95% of patients will achieve 85% of mCD39 occupancy in monocytes during the whole dosing interval (100% of the duration); At 3000 mg q4w, 91.8% of patients reaching 85% of mCD39 occupancy with 100% duration of exposure. 2000 mg q3w can achieve >90% patient coverage but not for Q4W (84% patient coverage). 2250 mg q4w however is expected to achieve about >90% patient coverage of 85% of mCD39 occupancy with 100% duration of exposure.

### Safety

Overall, 57 patients received treatment (IPH5201, n=38; IPH5201+durvalumab, n=19). No DLTs were observed during dose escalation. Treatment-emergent adverse events (TEAEs) occurred in 96.5% of patients; 40.4% had grade ≥3 TEAEs. The most common TEAEs were fatigue (28.1%), decreased appetite (26.3%), infusion-related reactions (21.1%), anaemia (19.3%) and tumour pain (19.3%). Treatment-related adverse events (TRAEs) occurred in 66.7% of patients (37/57 (64.9%) related to IPH5201; 11/19 (57.9%) related to durvalumab). 10.5% of patients had grade 3 TRAEs; there were no grade 4 TRAEs. The most common TRAEs were infusion-related reactions (21.1%), fatigue (17.5%), nausea, arthralgia, tumour pain and pruritus (8.8% each). There were no deaths due to treatment. Maximum tolerated dose (MTD) was not reached. No significant correlation of dose with incidence of Treatment-emergent adverse events of at least grade 3 was observed.

### Pharmacokinetics and Pharmacodynamics

CD39 occupancy by IPH5201 in samples from patients was determined by assessing free soluble CD39 and free membrane bound CD39.

To assess free soluble CD39, a monoclonal anti-CD39 antibody that competes with IPH5201 is coated on a microtiter plate and thereby permits free CD39 to be captured. A second non-competing anti-CD39 monoclonal antibody that carries a biotin label is used to bind to a site on CD39 distinct from the capture antibody. Bound biotin molecules are detected by addition of streptavidin horseradish peroxidase (SA-HRP) conjugate. Tetramethylbenzidine enzyme substrate is added to generate a colorimetric reaction that is measured at a wavelength of 450 nm. The concentration of free CD39 in a sample is determined by interpolation from a standard curve using a four parameter curve fit with 1/Y² weight value relating the color intensity to the concentration of CD39.

To assess free CD39 on monocytes and B cells, blood samples are spiked with an antibody having the VH and VL of IPH5201 and a mouse Fc domain and labelled with a fluorochrome. A second non-competing anti-CD39 monoclonal antibody labelled with a distinct fluorochrome is used to bind to a site on CD39 distinct from the capture antibody. CD14, CD19 and CD45 are detected by incubation with anti-CD14, CD19 and CD45 monoclonal antibodies each labelled with a distinct fluorochrome. Binding is detected by flow cytometry.

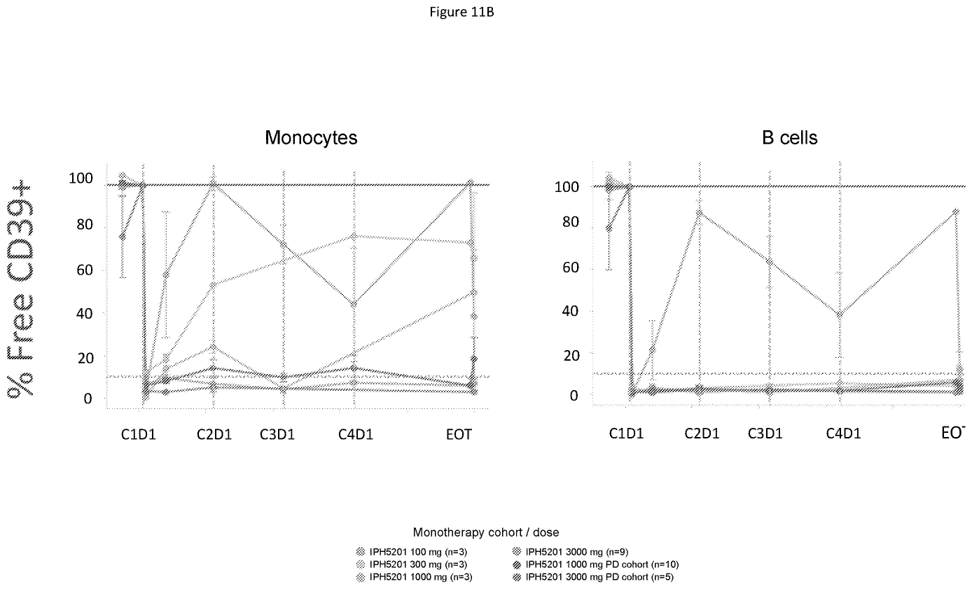

Results are shown in FIGS. 11A and 11B. C1D1 indicates cycle 1 pre-dose; C1D2 indicates cycle 1 post-dose; C1D8 indicates cycle 1 day 8; C2D1 indicates cycle 2 day 1; C3D1 indicates cycle 3 day 1; EOT indicates end of treatment; LLOQ, lower limit of quantitation; V1, visit 1 (at screening). FIG. 11A shows that IPH5201 saturated binding of soluble CD39 at ≥300 mg. Each line represents data from one patient. Increase in total soluble CD39 (not shown) is consistent with increased stabilisation of soluble CD39 by antibody binding. Similar trends were observed in combination treatment with durvalumab. FIG. 11B shows that IPH5201 saturated binding of membrane-bound CD39 on immune cells at 3000 mg. In monocytes (right hand panel), the 100 mg IPH5201 dose led to a full rebound of free CD39 at C2D1, the 300 mg IPH5201 dose to a partial rebound of free CD39 at C2D1. In B-cells only the 100 mg IPH5201 dose led to a rebound of free CD39 at C2D1.

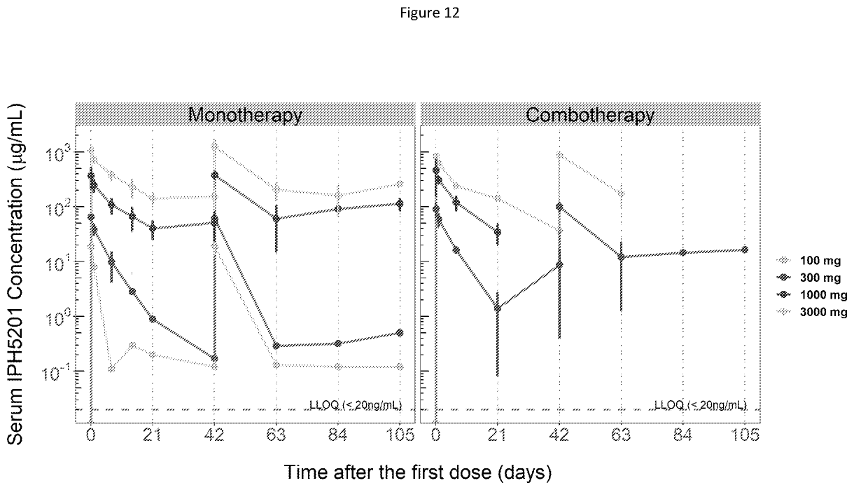

FIG. 12 shows the pharmacokinetics (PK) of IPH5201 either as monotherapy (left hand panel) or as combination therapy with durvalumab (right hand panel), with the time after first dose in days on the x-axis and serum IPH5201 concentration on the Y-axis. On the left hand panel, curves from bottom to top represent 100 mg, 300 mg, 1000 mg and 3000 mg doses of IPH5201; on the right hand panel curves from bottom to top represent 300 mg, 1000 mg (up to the 21 day time point) and 3000 mg doses of IPH5201. The PK of IPH5201 was non-linear at ≤300 mg and linear at ≥1000 mg. Free soluble CD39 was above the lower limit of quantitation at cycle 1, day 2 or later in 6/40 patients (n=2, 100 mg; n=1, 1000 mg; n=3, 3000 mg).

Tumour samples were obtained from patients with pancreatic cancer and assessed for CD39 enzyme activity. CD39 enzyme activity was assessed using the Wachstein-Meisel assay to detect the presence of phosphates hydrolyzed from ATP due to enzymatic activity of CD39. Enzymatic activity was assessed by semi-quantitative scoring methodology for tumour, stroma and vasculature. The pathologist conducted an overall assessment to determine whether a significant decrease was present in Day 15 compared with screening biopsies. The table below shows the tumoral CD39 enzymatic activity decreased in 5/8 patients with available samples. The 3000 mg dose of IPH5201 showed a decrease in enzymatic activity.

| | | | Significant overall |
|---|---|---|---|
| | | | decrease in CD39 |
| Patient | Cohort | Cancer | enzymatic activity? |
| 1 | 1000 mg | NSCLC | No |
| | | (squamous) | |
| 2 | 1000 mg | NSCLC | N/A (no baseline) |
| | | (squamous) | |
| 3 | 1000 mg | Pancreatic | Yes |
| 4 | 3000 mg | Pancreatic | Yes |
| 5 | 3000 mg | Pancreatic | Yes |
| 6 | 3000 mg | Pancreatic | Yes |
| 7 | 3000 mg | NSCLC | Yes |
| | | (squamous) | |
| 8 | 1000 mg + durva | Pancreatic | No |

In summary, the PK/PD modelling and simulation predicted a proposed dose of 3000 mg Q3W or Q4W, with or without durvalumab, would achieve ≥85% mCD39 occupancy on monocytes in >90% of patients, and maintain sufficient exposure time (i.e., 100% of duration of the dose interval) at steady state. A dose for IPH5201 of 3000 mg administered Q3W or Q4W can therefore provide advantages for use in both monotherapy and combination therapy with durvalumab, including decrease in risk of errors in dosing and the possibility for administration on the same days as durvalumab.

### Example 7: Design of a Phase 2 Single Arm Study Design of IPH5201 in Resectable NSCLC

Based on the results of the foregoing results, a phase 2 single arm clinical trial study of IPH5201 in resectable NSCLC was designed.

The key eligibility criteria are resectable (stage II/IIIA) NSCLC, no prior treatment, and Eastern Cooperative Oncology Group (ECOG) Performance Status (PS) of 0 or 1, all CPS status for PDL1 expression, EGFR/ALK wild type.

The primary endpoints are pathological complete response (pCR) and safety. The secondary endpoints: surgery feasibility; major pathological response (mPR); objective response rate (ORR); median and landmark (12 month) event free survival (EFS), disease free survival (DFS) and overall survival (OS); as well as assessment of PK and anti-drug antibodies (ADA) of IPH5201. The study will include a safety run-in in the first six patients.

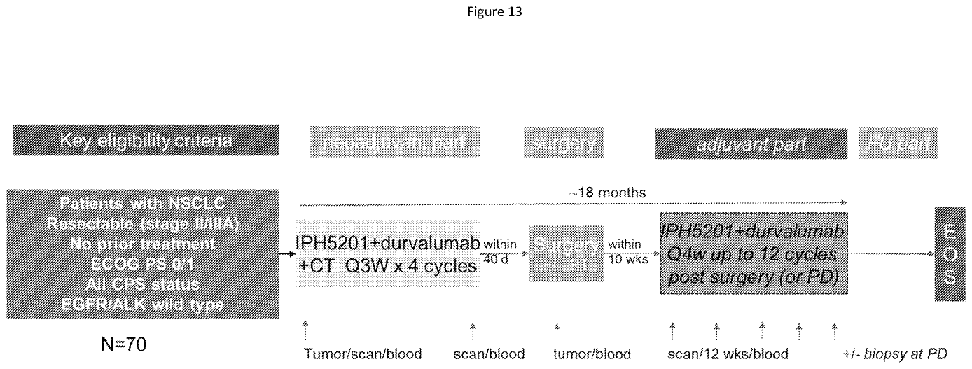

FIG. 13 shows a schematic of the treatment regimen for the treatment. As neoadjuvant therapy, IPH5201 and durvalumab are administered at Q3W for 4 cycles at a fixed dose of 3000 mg IPH5201 (2250 mg can be used as an alternative) and durvalumab at a fixed dose of 1500 mg, in combination with chemotherapy (CT). CT includes the carboplatin/paclitaxel combination, the cisplatin/gemcitabine combination, the cisplatin/pemetrexed combination, or the carboplatin/pemetrexed combination. Within 40 days of last dose of study drug in the neoadjuvant therapy, patients undergo surgical tumor resection with our without additional radiotherapy. Within 10 weeks of surgery, patients receive adjuvant therapy with IPH5201 and durvalumab for Q4w for up to 12 cycles post-surgery or until progressive disease, at a fixed dose of 3000 mg IPH5201 (or 2250 mg if used as an alternative) and durvalumab at a fixed dose of 1500 mg.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar references are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment herein using terms such as “comprising”, “having,” “including,” or “containing” with reference to an element or elements is intended to provide support for a similar aspect or embodiment herein that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1-31. (canceled)

32. A method of treating a cancer in a patient in need thereof, the method comprising administering to the patient an effective amount of a treatment regimen comprising an anti-CD39 antibody and an anti-PD(L)1 antibody, optionally wherein the anti-CD39 antibody comprises (a) a HCDR1, HCDR2 and HCDR3 of SEQ ID NO: 2, SEQ ID NO: 3 and SEQ ID NO: 4, respectively, and (b) an LCDR1, LCDR2 and LCDR3 sequence of SEQ ID NO: 5, SEQ ID NO: 6 and SEQ ID NO: 7, respectively, wherein the treatment regimen is a neoadjuvant therapy.

33. The method of claim 32, wherein the neoadjuvant therapy treatment regimen further comprises a chemotherapeutic agent.

34. The method of claim 32, wherein the treatment regimen is a neoadjuvant therapy and an adjuvant therapy.

35. The method of claim 32, wherein the anti-CD39 antibody is administered at a fixed dose of 3000 mg.

36. The method of claim 32, wherein the anti-CD39 antibody is administered once every 3 weeks as a neoadjuvant therapy.

37. The method of claim 32, wherein the anti-CD39 antibody and the anti-PD(L)1 antibody are each administered once every 3 weeks as a neoadjuvant therapy.

38. The method of claim 32, wherein the anti-CD39 antibody and an anti-PD(L)1 antibody are administered on day 1 of a 3 week cycle as a neoadjuvant therapy, for four cycles.

39. The method of claim 37, wherein the anti-CD39 antibody is administered once every 4 weeks as adjuvant therapy.

40. The method of claim 37, wherein the anti-CD39 antibody and the anti-PD(L)1 antibody are each administered once every 4 weeks as adjuvant therapy.

41. The method of claim 32, wherein the treatment regimen comprises: (a) administering to the patient, before surgical tumor resection, an effective amount of an anti-CD39 antibody, an anti-PD(L)1 antibody and a chemotherapeutic agent, (b) administering to the patient, after surgical tumor resection, an effective amount of an anti-CD39 antibody and an anti-PD(L)1 antibody.

42. The method of claim 32, wherein the anti-CD39 antibody comprises a heavy chain comprising the amino acid sequence of SEQ ID NO: 10 and a light chain comprising the amino acid sequence of SEQ ID NO: 11.

43. The method of claim 32, wherein the anti-PD(L)1 antibody comprises a heavy chain comprising the amino acid sequence of SEQ ID NO: 14 and a light chain comprising the amino acid sequence of SEQ ID NO: 15.

44. The method of claim 32, wherein the cancer is surgically resectable.

45. The method of claim 32, wherein the cancer is a lung cancer.

46. The method of claim 45, wherein the cancer is a non-small cell lung cancer.

47. The method of claim 32, wherein the patient has not received prior treatment for the cancer.

48. The method of claim 46, wherein the cancer is EGFR/ALK wild type.

49. A method of administering to a human patient an anti-CD39 antibody comprising a heavy and light chain respectively comprising the amino acid sequences of SEQ ID NOS: 8 and 9, wherein the anti-CD39 antibody is administered once every 3 weeks or once every 4 weeks at a fixed dose of 3000 mg.

50. The method of claim 49, wherein the anti-CD39 is administered in combination with an anti-PD(L)1 antibody, optionally wherein the anti-PD(L)1 antibody is administered on day 1 of the 3 week cycle for one or more cycles, and on day 1 of the 4 week cycle for one or more cycles, optionally wherein the anti-PD(L)1 antibody is durvalumab, optionally wherein durvalumab is administered at a fixed dose of 1500 mg.

51. A kit for treating a cancer or a tumor in a human patient, the kit comprising:
(a) one or more vials comprising an anti-CD39 antibody comprising the H-CDR1, H-CDR2 and H-CDR3 domains of a heavy chain variable region having the sequence set forth in SEQ ID NO: 8, and the L-CDR1, L-CDR2 and L-CDR3 domains of a light chain variable region having the sequence set forth in SEQ ID NO: 9; and/or
(b) one or more vials comprising durvalumab; and
(c) optionally, instructions for using said anti-CD39 antibody and/or durvalumab.

52. The kit of claim 51, wherein each vial of anti-CD39 antibody comprises 375 mg of anti-CD39 antibody.
